(19) United States
(12) Patent Application Publication
(10) Pub. No.: US 2002/0102661 A1
(43) Pub. Date: Aug. 1, 2002

(54) Compositions and processes of enzymatically modified polysaccharides

(76) Inventors: Ronald Busink, Bennekom (NL); Monique Verbeek, Acacialaan (NL); Alfred J. Haandrikman, Amersfoort (NL); Michaela Hofbauer, Roosendaal (NL); Henny W.C. Vaessen-Van Hoven, Voorthuizen (NL)

Correspondence Address: Hercules Incorporated, Hercules, Plaza, 1313 North Market Street, Wilmington, DE, 19894-0001, US

(21) Appl. No.: 09/918,891

(22) Filed: Jul. 31, 2001

Related U.S. Application Data
(60) Provisional application No. 60/222,645, filed on Aug. 3, 2000.

(51) Int. Cl. C12P 19/04; C08B 37/00

(52) U.S. Cl. 435/101; 536/114

(57) ABSTRACT

A composition and process for improving the oxidation of a galactose containing polysaccharide in the papermaking industry. The composition includes an aqueous solvent, a galactose containing polysaccharide, a one electron oxidant and a hydrogen peroxide remover. The process includes adding a composition for use in the papermaking industry comprising galactose containing polysaccharide, galactose oxidase, a one electron oxidant, and a hydrogen peroxide remover.

### CROSS-REFERENCE TO RELATED APPLICATIONS

This Application is related to U.S. Provisional Patent Application Ser. No. 60/222,645, filed 3 Aug. 2000, from which priority is claimed.

### BACKGROUND OF THE INVENTION

1. Field of the Invention

The present invention is directed to improved compositions and processes which enhance the activity level of the enzyme galactose oxidase for use in industrial and/or large scale applications. Specifically, the present invention is directed to compositions and processes which improve the activity level of galactose oxidase by the substantially continuous activation of galactose oxidase from its inactive form to its active form. More specifically, the present invention is directed to processes and compositions which improve the activity level of galactose oxidase by adding a one electron oxidant which substantially continuously activates galactose oxidase and by adding a hydrogen peroxide remover to decompose the hydrogen peroxide which is formed as a co-product in the oxidation of alcohols by galactose oxidase. The present invention is directed, by way of nonlimiting example, to improved compositions and processes for the oxidation of primary alcohols such as galactose containing compounds including polysaccharides, such as, for example, carbohydrate gums such as guar gum, for use in various applications including without limitation the papermaking industry, cross-linking agents, film-forming applications as adhesives, binding agents in self sustaining films and the like, drug delivery systems, tertiary oil recovery, drilling fluids, blood plasma volume expanders, and the like.

2. Background of the Invention and Related Art

The enzyme galactose oxidase (GaOx) is well recognized. The use of galactose oxidase is known in such reactions as the oxidation of primary alcohols, including galactose containing polysaccharides such as guar gum. Polysaccharides, such as guar gum, are known to have a variety of uses and the commercial value of carbohydrate gums is well recognized. A general discussion of carbohydrate gums is presented in R. L. Whistler, J. N. BeMiller, (Eds.) Industrial gums: polysaccharides and their derivatives. 1993, Academic Press Inc. San Diego, Calif. 92101, the entire contents of which is hereby incorporated by reference as though set forth in full herein.

The product of the oxidation of aqueous solutions of guar gum and other galactose bearing polysaccharides using galactose oxidase enzyme was disclosed by F. J. Germino in U.S. Pat. No. 3,297,604, the entire contents of which is hereby incorporated by reference as though set forth in full herein. Germino discloses the use of the oxidized products of polysaccharides in the manufacture of paper as well as for use to cross-link polyamino polymers, polyhdroxy polymers, and proteins. Germino further discloses the use of the oxidized polysaccharide as an intermediate or precursor to many carbonyl-reactions, including for use as a cross-linking agent for a broad range of natural or synthetic polymers and various film forming applications such as for use as an adhesive or film forming agent or binding agent in self-sustaining films. Germino discloses, for example, that the oxidation reaction product of polymers of galactomannan may be used in the manufacture of paper or tobacco sheets.

C. W. Chiu, et al., EP 281,655-B, the entire contents of which is hereby incorporated by reference as though set forth in full herein, discloses additional uses for oxidized polysaccharides. For example, Chiu discloses the syntheses of various starch aldehydes, such as aldehyde containing heteropolysaccharides, which may be of use as crosslinking agents and in the paper and textile industries. Specifically, Chiu discloses a hydroxypropyl galactoglycoside starch ether oxidized with galactose oxidase and catalase.

C. W. Chiu, et al., U.S. Pat. No. 5,554,745, and U.S. Pat. No. 5,700,917, the entire contents of which are hereby incorporated by reference as though set forth in fill herein, discloses (1) the preparation of cationic galactose containing polysaccharides and (2) the enzymatic oxidation in aqueous solution of the cationic galactose containing polysaccharides with galactose oxidase. The oxidized cationic polysaccharides are disclosed to improve the strength characteristics of paper and for use in the textile industry. As discussed in Chiu, it is known in the papermaking industry to use galactose oxidase to modify galactose containing polysaccharides. Specifically, the galactose oxidase modifies the galactose containing polysaccharide by introducing aldehyde groups into the polysaccharides. It is further known to use the modified polysaccharides in paper industry as paper additives, which improves various properties of the paper such as the strength of the paper and the ability of the paper to retain color.

Polysaccharides are further known to be useful in a variety of other industrial applications, including, as described by D. F. DeMasi, et. al., U.S. Pat. No. 4,453,979, the entire contents of which is incorporated by reference as though set forth in fill herein, the use of hydrophilic gums in such the cosmetic, pharmaceutical and personal care products industries by acting as thickeners, binders, stabilizers, protective colloids, suspending agents and flow control agents. The oxidized polysaccharides can also be used in industrial application such as tertiary oil recovery, drilling fluids, blood plasma volume expanders, and the like. M. Yalpani, et. al., Some Chemical and Analytical Aspects of Polysaccharide Modifications, J. of Polymer Science, Vol.20, 3399-3420 (1982), the entire contents of which is hereby incorporated by reference as though set forth in full herein.

The polysaccharides of the present invention are oxidized by galactose oxidase. Galactose oxidase has been given EC Number 1.1.3.9 and may be produced by the fungus Dactylium dendroides, recently renamed as Fusarium ssp., as described by Z. Ogel et.al Cellulose-triggered sporulation in the galactose oxidase producing fungus Cladabotyrum (Dactylium) dendroides NRRC 2903 and its reidentification as a species of Fusarium., Mycol. Res. 98(4), 474-480 (1994).

Without being bound by theory, galactose oxidase is believed to be present in three oxidative states: The active oxidized form contains a Cu²⁺ and a tyrosine radical in the active site, the reduced form which results from the two electron redox reaction by which a primary alcohol is converted to an aldehyde (Cu⁺, tyrosine), and an intermediate semi-form containing Cu²⁺ and tyrosine. The latter form is catalytically inactive. P. F. Knowles, et al. Galactose Oxidase. Perspectives on Bioinorganic Chemistry. Vol.2 (1993) pps. 207-244, the entire contents of which is hereby incorporated by reference as though set forth in full herein. And, in fact, the active form of the enzyme, which may contain the tyrosine free radical, spontaneously decays to the semi, inactive form.

The radical decay mechanism of galactose oxidase has only recently been fully described. It is believed, without being bound by theory, that the tyrosine free radical of the enzyme is unstable and decays via an electron transport chain built into the enzyme structure as a protective pathway. This mechanism is believed to establish an equilibrium of approximately ninety-five percent (95%) inactive or semi form of the enzyme and approximately 5% active enzyme. Saysell, et al., Kinetic Studies on the Redox Interconversion of Goase(semi)and Goase(ox) Forms of Galactose Oxidase with Inorganic Complexes as Redox Partners (1997) Vol. 36, Inorg. Chem, pp. 4520-4525, the entire contents of which is hereby incorporated by reference as though set forth in fill herein. The equilibrium is reached approximately three (3) hours after complete activation of the enzyme with an oxidant like ferricyanide. Id.

The reduced form of the enzyme can be reoxidized to the fully oxidized form by molecular oxygen, the reaction product of which is hydrogen peroxide. However, molecular oxygen does not appear to be able to carry out the one electron oxidation from the inactive semi-form to the active form. Rather, the oxidation must be achieved by other means. The literature describes several different techniques for oxidizing the semi (inactive) form of galactose oxidase to the active form. It is possible, for example, to obtain a fully activated galactose oxidase from the equilibrium mixture by a chemical oxidation, e.g., by ferricyanide, H₂IrCl₆, [Co(phen)₃]³⁻, [Co(dipic)₂]⁻. Saysell, et al., Kinetic Studies on the Redox Interconversion of Goase(semi and Goase(ox) Forms of Galactose Oxidase with Inorganic Complexes as Redox Partners (1997) Vol. 36, Inorg. Chem, pp. 4520-4525; P. Knowles and N. Ito, Perspectives in Bioorganic Chemistry, 1993, 2:207-241, the entire contents of which are hereby incorporated by reference as though set forth in full herein. Further, U.S. Pat. No. 4,220,503, to Johnson, indicates that ferricyanide can be used to stabilize the active form of the enzyme for electrochemical analysis.

Chemical oxidants such as ferricyanide are useful for a single activation cycle to obtain substantially 100% active enzyme for analytical purposes. However, in a synthetic application, these oxidants are consumed in every enzyme reactivation cycle. Thus, a large excess of oxidant with respect to galactose oxidase must be added to the system to achieve long term activation.

It is also possible to interrupt the deactivating radical decay pathway naturally present in the enzyme by protein engineering. It has been shown, for example, that the substitution of either of the two cysteine residues in the enzyme structure stops the radical decay. H. S. Ogilvie, 1998, “Protein Engineering of Galactose Oxidase”, PHD-thesis, Department of Biochemistry and Molecular Biology, University of Leeds, United Kingdom, the entire contents of which is hereby incorporated by reference as though set forth in full herein. The interruption of the electron pathway leads to long term instability of the protein primary structure under analytical circumstances, but provides an enzyme which has an activity level of approximately one-hundred percent (100%), for a prolonged time period.

Alternatively, it is possible to oxidize galactose oxidase electrochemically, in the presence of a suitable mediator, e.g., ferricyanide or ferrocene derivatives. T. Yamaguchi, Y. Murakami, K Yokoyama, H. Komura, E. Tamiya, Denki Kagaku 63 (1995) 1179-1182; T. Yamaguchi, E. Tamiya, Denki Kagaku 62 (1994) 1258-1259. The mediator is responsible for the electron transfer between the enzyme and the anode, which is not possible directly. However, in contrast to the stoichiometric use of ferricyanide as mentioned above, in the electrochemical system, the anode is the stoichiometric oxidant. Thus, the mediator need only be present in catalytic amounts and is not consumed during the course of the reaction. A. Petersen, E. Steckhan, Bioorganic & MedicinalChemistry,7 (1999) 2203-2208, the entire contents of which is hereby incorporated by reference as though set forth in full herein.

It has been demonstrated that the activity of galactose oxidase is somewhat enhanced in the presence of horseradish peroxidase. For example, an increased activity of galactose oxidase in the presence of peroxidase under assay conditions has been described by Kwiatkowski, et al., in On The Role Of Superoxide Radical In The Mechanism Of Action Of Galactose Oxidase, Vol. 53 No. 3 (1973) Biochemical and Biophysical Research Communications, the entire contents of which is hereby incorporated by reference as though set forth in full herein. Although Kwiatkowski et al., indicated that adding catalase to galactose oxidase, peroxidase and a substrate did not retard the oxidation of the substrate, they found no advantages to such a system.

It has also been shown that in radioactive labelling of glycolipids, the use of catalase and horseradish peroxidase in conjunction with galactose oxidase will assist in increasing the specific activity of gangliosides because the addition of both catalase and horseradish peroxidase to galactose oxidase increases the amount of GalNAc oxidized by galactose oxidase. Novak, et al., Preparation of Radiolabeled BM2 and GA2 Gangliosides, Journal of Lipid Research Vol. 20:678 (1979). This article sets forth a procedure to purify two types of gangliosides found in the brain of a patient who died from Sandhoff's disease. The article also sets forth a procedure to increase the specific activity of radiolabeled gangliosides, which are produced by galactose oxidase oxidation and successive reduction by tritiated NaBH4. The yield of tritium incorporation can be increased by using a peroxidase, catalase and galactose oxidase for the oxidative reaction.

The increased catalytic activity of galactose oxidase in the presence of a peroxidase and catalase has also been shown by Radin, et al., in The Use of Galactose Oxidase in Lipid Labelling, J. Lipid Res., Vol. 22:536-541, (1981). In this publication, Radin, et al., used galactose oxidase to oxidize lipids which included galactose or galactosamine and then, after oxidation, reduced the compound. Catalase and peroxidase were added to improve the yields of aldehyde lipids and it was found that the addition of both catalase and peroxidase increased the rate of oxidation performed by galactose oxidase. This method was used in the study of surface membranes by labelling various substrates of galactose oxidase.

However, none of the prior art that combines peroxidase, catalase and galactose oxidase utilizes the combination in a reaction with polysaccharides. Moreover, none of the prior art discusses the low activity level of galactose oxidase and suggests a method or process to increase the activity level of galactose oxidase in any commercial application or otherwise by continuously activating the inactive form of galactose oxidase to the active form of the enzyme. While it was recognized that adding catalase and peroxidase to galactose oxidase and substrate may, at least in some instances, increase the activity level of galactose oxidase, it was not recognized to use galactose oxidase more efficiently in larger scale synthetic applications.

In fact, the state of the prior art appears to indicate that, in synthetic systems, including, without limitation, the production of additives for the papermaking industry, where no activator for galactose oxidase is incorporated, the enzyme is present at effectively only about 5% activity level of the total level that is present and that will be used for the synthesis. Further, in recent publications regarding the use of guar in synthetic systems, there is no reference to increasing the activity level of galactose oxidase by continuously or substantially continuously activating galactose oxidase by using a one electron oxidant, such as peroxidase or laccase, with a hydrogen peroxide remover, such as catalase, with galactose oxidase. Donnelly describes that the dual enzyme system Catalase and Galactose oxidase can be used for the oxidation of guar gum to produce products with various viscosities or gels under mild conditions and with low toxicity reagents. M. J. Donnelly, Viscosity control of guar polysaccharide solutions by treatment with galactose oxidase and catalase enzymes, In: C. Burke (Ed.) Carbohydrate Biotechnology Protocols, 1999, Humana Press, Totowa (N.J.), pp.79 B 88; Further, galactose oxidase in combination with catalase was used to oxidize guar to its poly-aldehyde derivative. The aldehyde material was used as starting material for the preparation of the poly-carboxylic acid by a second oxidation reaction. The latter product was studied as thickening agent with altered rheological properties. Frollini et al. (Carbohydrate Polymers 27 (1995) 129-135), the entire contents of which are hereby incorporated by reference as though set forth in full herein. Further, galactose oxidase in combination of catalase was used to prepare oxidized guar gum and locust bean gum as precursors for various chemically modified polysaccharides. The aldehyde functionality was used to attach various functional groups via reductive amination, oxidation, and reduction. M. Yalpani, et. al., Some Chemical and Analytical Aspects of Polysaccharide Modifications, J. of Polymer Science, Vol.20, 3399-3420 (1982).

However, the applications described thus far have suffered from the lack of measures taken to induce and/or maintain a good part or high percentage of the enzyme in its active form, especially during the full course of the synthesis. Therefore, there is a need to have a fully active, or at least a more active, enzyme in the reaction in an industrial application or during a full scale operation, especially during the entire length of the reaction. For example, there is a need in the production of papermaking chemicals, as well as the other applications listed above, to have a more active enzyme during the course of the reaction.

Generally, it is counterintuitive in industrial applications, such as the production of papermaking chemicals, binding agents and the like, to add for reasons of cost reduction an additional enzyme, to a full scale reaction. Conventional wisdom on this point would indicate that adding an additional substance, such as an additional enzyme, to a known reaction would increase the overall cost of the process: One skilled in the art of the production of papermaking chemicals, adhesives and the like would not consider adding an additional component, such as a third enzyme, to a process involving the oxidation of a polysaccharide.

There is, thus, a need in the art to provide reaction conditions having enhanced proportions of active enzyme.

### SUMMARY OF THE INVENTION

In view of the foregoing, the present invention is directed toward continuously or substantially continuously enhancing the activity level of an oxidizing agent, such as galactose oxidase, for use in industrial applications, such as the production of additives for the papermaking industry, binding agents, cross-liking agents and the like, and in full scale chemical synthesis operations.

The present invention is intended to include and encompass any and all industrial applications or full scale chemical synthesis operations which would benefit from the ability to use a fully active, or substantially fully active, enzyme during the course of the reaction, including, without limitation, the production of materials for use in the papermaking industry, the tobacco industry, and the use of the oxidized products of polysaccharides as an intermediate or precursor to many carbonyl reactions, including for use as a cross-linking agent, for use in film forming applications, or for use as a rheology modifying agent.

Conventional wisdom teaches away from the addition of a third enzyme to the oxidation reaction involving galactose oxidase. As discussed above, one skilled in the art of the various industrial applications such as, for example, producing adhesives, films, cross-linking agents, or additives for the papermaking industry, would usually not consider adding an additional component, such as a third enzyme to the oxidation of polysaccharides because of the added cost of an additional component, such as a third enzyme, would represent. However, the present invention produces the surprising result of decreasing the cost of an industrial application by adding an additional component, such as a third enzyme to the oxidation of polysaccharides.

The present invention is further directed to increasing the wet and dry paper strength by increasing the aldehyde content of a galactose containing polymer used as papermaking additive by increasing the activity level of galactose oxidase by continuously, or substantially continuously, activating galactose oxidase from the semi or inactive form to the active form by using a one electron oxidant or oxidizing agent in the presence of hydrogen peroxide remover.

Further, according to the present invention, there are provided paper products having improved strength characteristics prepared by using the combination of galactose containing polymers, galactose oxidase, a one electron oxidant and a hydrogen peroxide remover. The present invention is further directed to more efficiently increasing both wet and dry paper strength by increasing the aldehyde content of the paper by increasing the activity level of galactose oxidase and, thus, using less galactose oxidase, by continuously or substantially continuously activating galactose oxidase.

Even more specifically, and by way of nonlimiting example, the invention may be directed to improved processes and compositions for oxidizing polysaccharides, and more particularly carbohydrate gums such as guar gum, by increasing the activity level of galactose oxidase by continuously or substantially continuously activating galactose oxidase from the semi form to the active form by using a one electron oxidant in the presence of hydrogen peroxide remover such as, for example, catalase.

In accordance with one aspect of the invention, a number of oxidative enzymes are able to act as one electron oxidants and catalyze the reaction of semi (inactive) galactose oxidase to active galactose oxidase, including without limitation peroxidases, such as horseradish peroxidase, laccase, and soybean peroxidase.

In accordance with another aspect of the present invention, the oxidative enzymes such as horseradish peroxidase, laccase and soybean peroxidase can be added in much smaller quantities, thereby being more efficient and cost effective to use these types of enzymes as one electron oxidants as opposed to the chemical oxidants, such as ferricyanide. The oxidative enzymes act as oxidative catalysts, thus only catalytic amounts with respect to galactose oxidase are needed to achieve the continuous, or substantially continuously, activation of galactose oxidase. Whereas the use of chemical oxidant, such as ferricyanide, requires a large excess of the chemical to achieve effective levels of oxidation.

According to one aspect of the invention, the one electron oxidants such as horseradish peroxidase, laccase and soybean peroxidase, may convert inactive galactose oxidase to the active form.

In accordance with another aspect of the present invention, a hydrogen peroxide remover may be added to the galactose oxidase, the one electron oxidant and the substrate. In accordance with yet another aspect of the present invention, the addition of catalase as the hydrogen peroxide remover may added to the galactose oxidase, the one electron oxidant and the substrate.

The present invention is further directed to processes for producing oxidized carbohydrate gums. Products produced according to these processes are also contemplated. The present invention includes processes whereby aggregates of oxidized guar gum or aggregates of oxidized guar gum derivatives can be prepared, stored, and subsequently dissolved in water without significantly affecting the molecular weight and the aldehyde content of the product.

The present invention is further directed to processes of recovering oxidized carbohydrate gum, and more particularly, oxidized guar gum and/or oxidized guar gum derivatives, from aqueous reaction mixtures. Products produced according to these processes are also contemplated.

The present invention is more particularly directed to processes for recovering oxidized carbohydrate gum from aqueous reaction mixtures, which mixtures may further comprise viscosity reducing agents. Products produced according to these processes are also contemplated.

The present invention is directed to a composition including galactose oxidase, galactose containing polysaccharide, a one electron oxidant, hydrogen peroxide remove and aqueous solvent. Further, the hydrogen peroxide remover may be an enzyme, such as catalase. Still further, the one electron oxidant may be an enzyme, such as, for example, horseradish peroxidase, laccase and soybean peroxidase.

In accordance with the present invention, the galactose containing polysaccharide may include at least one of carbohydrate gums, pectins and cellulosics. Still further, the galactose containing polysaccharides may include carbohydrate gums, which may include at least one of polygalactomannan gums or their ether derivatives, arabinogalactan gums or their ether derivatives, galactoglucomannan hemicelluloses or their ether derivatives, carubin, lichenan, tamarind and potato galactan, polygalactoglucans, polygalactoglucomannans and polygalactan gums.

In accordance with the present invention, the carbohydrate gum may include polygalactomannan gum, which may, more particularly, include at least one of locust bean gum, guar gum, tamarind gum, gum arabic, tara and fenugreek. Still further, the polygalactomannan gum may include guar gum.

Still further, the carbohydrate gum may include polygalactan gum, which can further include at least one of carrageenans and alginates.

In accordance with the present invention, the one electron oxidant may include a chemical oxidant, which can include at least one of ferricyanide, H₂IrCl₆, [Co(phen)₃]³⁻, [Co(dipic)₂]⁻.

Still further, the one electron oxidant may include ferricyanide and the hydrogen peroxide remover may include catalase.

Still further, the polysaccharide may include guar; the one electron oxidant may include horseradish peroxidase and the hydrogen peroxide remover may include catalase. In accordance with the present invention, the polysaccharide composition of the present invention may be solid. And, still further, the composition ma be re-solubilized.

The present invention contemplates the composition further including paper fiber, or natural or synthetic polymers, or plasma.

The present invention is directed towards a process or method for oxidizing a galactose oxidase substrate containing at least one alcohol group convertible to an aldehyde in an industrial application including reacting, in an aqueous solvent, the substrate, galactose oxidase, one electron oxidant capable of activating the galactose oxidase and hydrogen peroxide remover, under conditions to oxidize the galactose oxidase substrate.

Still further, the hydrogen peroxide remover may include catalase. Still further, the galactose oxidase substrate may include a polysaccharide, such as, for example, at least one of carbohydrate gums, pectins and cellulosics.

In accordance with the present invention, the carbohydrate gum may include at least one of polygalactomannan gums or their ether derivatives, arabinogalactan gums or their ether derivatives, galactoglucomannan hemicelluloses or their ether derivatives, carubin, lichenan, tamarind and potato galactan, polygalactoglucans, polygalactoglucomannans and polygalactan gums.

The present invention further includes a composition wherein the carbohydrate gum may be polygalactomannan gum, which may include at least one of locust bean gum, guar gum, tamarind gum, gum arabic, tara and fenugreek. Still further, the polygalactomannan gum may be guar gum. Still further, the carbohydrate gum may include polygalactan gum, which may include at least one of carrageenans and alginates.

In accordance with the present invention, the one electron oxidant may include at least one enzyme, such as, for example, horseradish peroxidase, laccase and/or soybean peroxidase.

The present invention contemplates the carbohydrate gum including guar, the one electron oxidant including soybean peroxidase and the hydrogen peroxide remover including catalase.

The present invention further contemplates the galactose oxidase substrate being a paper strength additive, or a binding agent for use in the paper industry.

Still further, the present invention contemplates the carbohydrate gum being guar, the one electron oxidant being horseradish peroxidase and the hydrogen peroxide remover being catalase. Still further, the present invention contemplates the one electron oxidant being a chemical oxidant, such as, for example, at least one of ferricyanide, H₂IrCl₆, [Co(phen)₃]³⁻,

The present invention further contemplates the one electron oxidant being ferricyanide and the hydrogen peroxide remover being catalase.

Still further, the invention further contemplates utilizing a solid or dried composition and re-solublizing the oxidized composition.

### BRIEF DESCRIPTION OF THE DRAWINGS

The present invention is further described in the detailed description which follows, in reference to the noted plurality of drawings by way of non-limiting exemplary embodiments of the present invention, and wherein:

FIG. 1 schematically illustrates a theoretical curve of the measurement of galactose oxidase activity in a BOM assay system as described in Example 4;

FIG. 2 is a graph showing the gas chromatography/mass spectrometry results (% aldehyde) for Sample A from Example 18.

FIG. 3 is a graph showing the gas chromatography/mass spectrometry results (% aldehyde) for Sample B from Example 18.

FIG. 4 is a graph showing the amount of dissolved Sample A from Example 18 using refractive index area as a measure of dissolved sample, with various temperatures and blender times.

FIG. 5 is a graph showing the amount of dissolved Sample B from Example 18 using Refractive Index area as a measure of dissolved sample, with various temperatures and blender times.

FIG. 6 is a graph showing the product of the Refractive Index area and the percent aldehyde groups of dissolved Samples A and B from Example 18, with various temperatures and blender times.

FIG. 7 is a graph showing sugar analysis compared with Size Exclusion Chromatography data (as Refractive Index area) of dissolved Sample A from Example 18, with various temperatures and blender times.

FIG. 8 is a graph showing the amount of dissolved Sample B from Example 18, with various mixers and with a temperature of 70° C. and a mixing time of 30 minutes.

FIG. 9 is a graph showing the amount of dissolved Sample B from Example 19 (0.1% sample in tap water) measured as Refractive Index area with various pH, 5 and 10 minutes mixing times, and a mixing temperature of 90° C. The pH values in parentheses are those values measured before mixing.

FIG. 10 is a graph showing the percent aldehyde groups in Sample B from Example 19 dissolved in tap water with various pH, 5 and 10 mixing times and a mixing temperature of 90° C. The pH values in parentheses are those values measured before mixing.

FIG. 11 is a graph showing the product of the Refractive Index area and the percent aldehyde groups of the dissolved Sample B of Example 19 (0.1% in tap water), as a function of pH, 5 and 10 minutes mixing times, and a mixing temperature of 90° C. The pH values in parentheses are those values measured before mixing.

### DETAILED DESCRIPTION OF THE INVENTION

The particulars shown herein are by way of example and for purposes of illustrative discussion of the embodiments of the present invention only and are presented in the cause of providing what is believed to be the most useful and readily understood description of the principles and conceptual aspects of the present invention.

The present invention is generally directed to increasing the amount of active, and thus useful, galactose oxidase present in a preparative reaction system, on a continuous, or substantially continuous basis. As the active form of galactose oxidase continuously decays to its inactive semi form during the course of a synthetic reaction due to the protective radical decay pathway built into the protein structure, it is necessary to also continuously reoxidize the semi form by a one electron oxidant to keep as much galactose oxidase as possible in its active form.

Having a fully active, or a substantially fully active enzyme throughout the full length of the reaction provides at least two advantages. First, because a higher percentage of the galactose oxidase is active during the synthetic application, less of the enzyme is required, thereby decreasing the overall cost of the process. Second, because the enzyme is continuously, or substantially continuously, activated by a one electron oxidant such as a peroxidase or a laccase, in the presence of a hydrogen peroxide remover such as catalase, only catalytic amounts, as opposed to stoichiometric amounts, of the one electron oxidant are required, thereby decreasing the overall cost of the synthetic application.

The present invention is intended to include and encompass any and all synthetic or industrial and/or full scale applications which would benefit from the ability-to use a fully active, or substantially fully active, enzyme during the course of the reaction, including, without limitation, the production of materials for use in the papermaking industry and the tobacco industry, and the use of the oxidized products of polysaccharides as an intermediate or precursor to carbonyl reactions, including for use as a cross-linking agent, in film forming applications and as a rheology modifying agent.

More particularly, the present invention is directed to compositions and processes for improving the activity level of galactose oxidase in various synthetic reactions, including production of additives for the papermaking industry.

The components of this invention, including the galactose oxidase, carbohydrate containing guar, one-electron oxidant and hydrogen peroxide remover, can be added anywhere in the process of papermaking, i.e., either before or after sheet formation. For example, they can be added before sheet formation (1) early during pulp preparation in the slurry chest or refiner chest, (2) in the machine chest or stock chest, (3) at other points in the wet end such as the fan pump or in-line mixers. They can also be added to the white water chest. Examples of addition after sheet formation are in the size press or even as a later coating process. The components can be premixed or added separately in any order. Preferable practice in the wet end, however, is to add the cationic polymer first.

In addition to dry strength, properties such as Z-direction tensile strength, Scott Bond Strength, Mullen burst, ring crush, tensile energy absorption (TEA) and fracture toughness can also be improved by using the combination of cationic water-soluble and/or water-dispersible polymers and oxidized galactose type of alcohol configuration containing polymer of the present invention.

Further, the present invention is directed to compositions and processes for increasing the efficiency of various synthetic reactions in the production of additives for the papermaking industry. Even more specifically, the present invention is directed to compositions and processes which include galactose oxidase, a galactose oxidase substrate, a one electron oxidant and a hydrogen peroxide remover. More particularly, the present invention is directed to produce a galactose containing polymer with a high level of aldehyde groups in an efficient manner, which can be used as strength additive in the papermaking process. The higher the aldehyde content of the additive, the higher are the strength levels of the paper made with the aldehyde containing additive.

The present invention is directed to continuously converting the inactive form of galactose oxidase (which spontaneously occurs) to the active form of galactose oxidase, such that a higher percentage of galactose oxidase is present in its active form and the overall activity level of galactose oxidase improves.

Converting the inactive form of galactose oxidase to the active form may be performed by using a one electron oxidant which is present in the synthetic system. Further, according to one particularly advantageous aspect of the invention, the activity of the galactose oxidase can be further enhanced by the addition of a hydrogen peroxide remover, which decomposes the coproduct hydrogen peroxide, which is being formed in stoichiometric amounts during the oxidation of alcohol to aldehyde. Hydrogen peroxide is a strong oxidizing agent, which is known to destroy enzymatic activity by disrupting the protein structure. Therefore, addition of a hydrogen peroxide remover like catalase has a protective effect on both galactose oxidase and the activating enzyme, for example, soy bean peroxidase.

When referring to components throughout this application, unless otherwise noted, reference to a component in the singular also includes combinations of the components.

The term “paper”, as used herein, is intended to broadly encompass “paper” in all forms, including sheet like masses of paper, molded or pre-formed paper, paper made from synthetic as well as natural sources, or any combination thereof.

The modification of starch and other polysaccharides by many different methods to produce various cation and aldehyde containing polysaccharides as well as cationic aldehyde containing derivatives is well known. Many of these modified polysaccharides have been used as paper additives to improve properties such as strength, drainage and pigment retention. And, in fact, an increase in the concentration of aldehydes is directly related to an increase in paper strength.

The term galactose oxidase, for purposes of the present invention, means that enzyme classified as E.C. No. 1.1.3.9 and those enzymes which function in a substantially similar manner, including, for example, glyoxal oxidase, (CAS Registration No. 109301-01-1). Also included are all enzymes, including those obtained through any form of genetic engineering with a catalytic domain which is substantially homologous with galactose oxidase or glyoxal oxidase. As used herein, the term galactose oxidase includes each of the three known oxidative states of galactose oxidase. Galactose oxidase has an approximate molecular weight of 68,000. Knowles and N. Ito, Perspectives in Bioorganic Chemistry, 1993, 2:207-241.

The term one electron oxidant, as used herein, is intended to include one electron oxidants alone and/or in combination. One electron oxidant, within the scope of the invention, means a substance capable of transforming the inactive form of galactose oxidase to its active form. One electron oxidant, as used herein, includes without limitation, the enzyme peroxidase (EC 1.11.1.7), such as horseradish peroxidase or soybean peroxidase, or the enzyme laccase (EC 1.10.3.2). Chemical one electron oxidants include all chemical one electron oxidants which are capable of converting the semi or inactive form of galactose oxidase to its active form, including, by way of nonlimiting example, ferricyanide, H₂IrCl₆, [Co(Phen)₃]³⁻, [Co(dipic)₂]⁻.

Without being bound by theory, it is believed that the one electron oxidant generates a tyrosyl radical in galactose oxidase. As discussed above, the active oxidized form of galactose oxidase contains a tyrosine radical in the active site. If enzymes are used as one electron oxidants, only catalytic amounts of the enzyme are required, because the stoichiometric oxidant (either oxygen or hydrogen peroxide) is already present in the reaction mixture in sufficient concentrations. If chemical one electron oxidants are used, they have to be added in at least stoichiometric amounts, preferably in excess with regards to galactose oxidase.

The term hydrogen peroxide remover, as used herein, is intended to include substances which remove or breakdown hydrogen peroxide. It is believed, without being bound by theory, that high levels of hydrogen peroxide may damage the protein structure of galactose oxidase and may inhibit or slow down the galactose oxidase reaction. Accordingly, it is beneficial to maintain the hydrogen peroxide concentration in the reaction medium as low as possible. For purposes of the present invention, hydrogen peroxide removers include, without limitation, catalases.

In addition to maintaining the concentration of hydrogen peroxide low to protect the galactose oxidase (and any other enzymes that may be present, including without limitation the one electron oxidants), the hydrogen peroxide remover can also play a role in providing the molecular oxygen that is needed by galactose oxidase to carry out the oxidation reaction. Galactose oxidase converts the oxidizable galactose type of alcohol configuration to the corresponding aldehyde group (thus producing oxidized galactose) by reducing oxygen to hydrogen peroxide. It is known in the art to provide the oxygen via aeration techniques, including bubbling oxygen gas through the solution.

In accordance with the present invention, however, the necessary amount of oxygen may be provided by adding hydrogen peroxide to the catalase containing reaction mixture, wherein the catalase breaks down the hydrogen peroxide into water and oxygen. The addition of oxygen to the reaction mixture by this method is more efficient because it avoids the oxygen transfer from the gas to the liquid phase. Preferably, to prevent the breakdown of the galactose oxidase and/or other enzymes present in the system, the hydrogen peroxide is gradually added to the reaction mixture. For optimum oxidation conditions, the addition velocity of the hydrogen peroxide solution is added in such a way that the dissolved oxygen concentration in the reaction mixture is maintained, or substantially maintained, at a consistent level. Preferably, the dissolved oxygen concentration is present at saturated or substantially saturated levels.

As used herein, the term galactose oxidase substrate is intended to include galactose oxidase substrates alone and/or in combination. For purposes of the present invention, galactose oxidase substrates include any compound containing one or more alcohol groups that galactose oxidase can convert into an aldehyde, including, by way of nonlimiting example, primary alcohols and polyols as mentioned in R. L. Root, Galactose Oxidase in stereospecific oxidation of primary alcohols, MS thesis, Texas A&M university, 1985, the entire contents of which is hereby incorporated by reference as though set forth in fill herein; and, by way of nonlimiting example, polysaccharides, such as, for example, carbohydrate gums.

Polysaccharides within the scope of this invention include, but are not limited to, polygalactomannan gums, pectins, polygalactoglucans; polygalactoglucomannans; and cellulose ethers such as hydroxyethylcellulose. Derivatives of all of these polysaccharides are also contemplated. In preferred embodiments, the polysaccharide or polysaccharide derivative is oxidized. Preferably, the polysaccharide comprises guar or its derivatives, and oxidized carbohydrate gum(s) preferably comprising oxidized guar or an oxidized guar derivative.

As used herein, the term carbohydrate gum is intended to include carbohydrate gums, alone and/or in combination. Carbohydrate gums within the scope of this invention include, but are not limited to, galactomannan gums or their ether derivatives, arabinogalactan gums or their ether derivatives, other gums or their ether derivatives, galactoglucomannan hemicelluloses or their ether derivatives and synthetically or enzymatically modified polymers. Preferred galactomannan gums are guar, locust bean, tara and fenugreek. Preferred arabinogalactan gums are arabic, larch and tragacanth gums. Preferred other gums are carubin, lichenan, tamarind and potato galactan. More preferred oxidizable galactose type of alcohol configuration containing polymers are the ether derivatives of guar gum such as anionic, amphoteric, hydroxypropyl, dihydroxypropyl and hydroxyethyl guar.

Synthetically or enzymatically modified polymers can be obtained by transferring an oxidizable galactose alcohol type of configuration to polymers. Glycosyl transferases or hydrolases can be used to transfer galactose from lactose unto e.g., polysaccharides to provide useful polymers for oxidation. Synthetic methods can also be used to attach the oxidizable galactose alcohol type of configuration.

Synthetically or enzymatically modified polymers can also be obtained by transferring an oxidizable primary alcohol group like ethylene glycol, propylene glycol or the like to a polysaccharide as in the case of hydroxyethyl cellulose, or to a synthetic polymer.

Generally, and by way of nonlimiting example, the present invention includes processes and compositions for improving the oxidation of galactose containing compounds by increasing the activity level of galactose oxidase. For example, the present invention includes processes and compositions for improving the oxidation of galactose containing polysaccharides or, more particularly, carbohydrate gums such as, for example, guar gum. Specifically, the present invention includes a composition containing polysaccharide, oxidizing agent which oxidizes the polysaccharide such as, for example, galactose oxidase, one electron oxidant, and hydrogen peroxide remover which enhances the continuous activation of galactose oxidase.

In accordance with the present invention, the amount of galactose oxidase present in a particular composition or reaction chamber would depend on the end product one is attempting to achieve. Moreover, in most commercial applications, there is no upper limit or maximum amount of enzyme that must be present to enable the reaction to proceed. However, based, at least in part, on the natural life of the enzyme, there usually is a minimum amount of enzyme, such as galactose oxidase, that must be present to achieve the level of oxidation (as measured in percentage of converted aldehyde) that is required by the particular industrial or large scale application. Additional enzyme may be added to speed up or accelerate the reaction; however, whether to add additional enzyme depends on the cost of adding additional enzyme versus the benefits that the additional enzyme will provide. In any event, one of ordinary skill in the relevant art would be able to select the concentration of galactose oxidase that should be present relative to the amount of galactose oxidase substrate that is being used and the end product one is attempting to obtain.

For example, in accordance with the present invention, the concentration of galactose oxidase in the aqueous mixture that includes guar gum or a guar gum derivative, may be greater than 1 IU, more preferably greater than 10 IU, and most preferably greater than 50 IU per gram of polysaccharide.

In accordance with the present invention, the amount of galactose oxidase substrate present would also depend on the product that one is attempting to achieve. For example, where galactose containing polysaccharides are being used as the galactose oxidase substrate, the concentration of polysaccharides may be greater than 0.1% (w/v), more preferably greater than approximately 0.3% (w/v) and most preferably greater than 0.6% (w/v).

In accordance with the present invention, the amount of one electron oxidant present in a particular composition or reaction chamber would depend on the end product one is attempting to achieve as well as the particular one electron oxidant chosen. One of ordinary skill in the relevant art would be able to select the concentration of one electron oxidant and would be able to select the particular one electron oxidant that should be used.

More specifically, using soybean peroxidase may also result in an increased activity level of galactose oxidase which results in very high oxidation levels of the substrate. In accordance with the present invention and as Tables 4, 5 and 10 show, it is possible to have soybean peroxidase as the one electron oxidant and to have a soybean peroxidase/galactose oxidase ratio of at least approximately 0.005 Units of soybean peroxidase to IU (International Units) of galactose oxidase. Preferably, it is possible to have the galactose oxidase activity level significantly increase when soybean peroxidase and galactose oxidase are present in ratios of at least approximately 0.01 U of soybean peroxidase to IU of galactose oxidase. It may be more preferable to have soybean peroxidase and galactose oxidase present in the ratio of at least approximately 0.05 (U/IU). It may be even more preferable to have soybean peroxidase and galactose oxidase present in the ration of at least approximately 0.1 (T/IU).

According to one aspect of the invention, and by way of nonlimiting example, using horseradish peroxidase as the one electron oxidant may result in an increased activity level of galactose oxidase which results in very high oxidation levels of the substrate. As Table 7 shows, it is possible to obtain an increased activity level of galactose oxidase, resulting in a higher degree of conversion as expressed in % aldehyde produced, when using a ratio of Units of horseradish peroxidase to International Units (I11) of galactose oxidase of at least approximately 0.006. Preferably, it may be possible to increase the activity level of galactose oxidase when preincubating, at room temperature, a ratio of Units of horseradish peroxidase to IU of galactose oxidase of at least approximately 0.01, even more preferably, the ratio may be at least approximately 0.05 Units of horse radish peroxidase to IU of galactose oxidase. Even more preferably, the ratio may be at least approximately 0.5 or 1 Units of horse radish peroxidase to IU of galactose oxidase.

In addition to using peroxidases as a one electron oxidant, it is also possible, according to the present invention, to use lacasses as a one electron oxidant to increase the activity level of galactose oxidase. The ratio between laccase and galactose oxidase (Units laccase:International Units galactose oxidase) should at least be greater than 0.01, more preferably greater than 0.05, even more preferably greater than 0.1, and most preferably greater than 1.

Further, in accordance with the present invention, if the reaction involves a chemical one electron oxidant, such as ferricyanide, then at least stoichiometric amounts of the chemical one electron oxidant must be added. It is preferable, moreover, to add an amount of chemical one electron oxidant that is sufficient to convert the galactose oxidase that has naturally decayed to its semi-or inactive form to its active form. Generally, this will require the chemical one electron oxidant to be present in amounts greater than a stoichiometric amount. It is even more preferable, moreover, to add an amount of chemical oxidant that will maintain, or substantially maintain, the redox potential of the reaction mixture at a level of at least 100 mV above the redox potential E°′ of the galactose oxidase (semi)—galactose oxidase (oxidized) redox couple throughout the length of the synthetic process. The redox potential E°′ varies with pH, ranging from a value of 0.38 V vs NHE (normal hydrogen electrode) at a pH of 8.5 to a value of 0.50 V vs NHE at a pH of 5.5.

The present invention is intended to include and encompass all chemical one electron oxidants which are capable of converting the semi or inactive form of galactose oxidase to its active form, including, without limitation, ferricyanide, H₂IrCl₆, [Co(phen)₃]³⁻, [Co(dipic)₂]⁻.

In accordance with the scope of the invention, a second substance may be added to the substrate-galactose oxidase-one electron oxidant system. According to the scope of the present invention, the second substance facilities or increases the ability of galactose oxidase to perform its oxidizing function by removing hydrogen peroxide which is formed as a coproduct in the oxidation of alcohols by galactose oxidase. These second substances include, without limitation, catalase.

It is believed, without being bound by theory, that high levels of hydrogen peroxide may damage the protein structure of galactose oxidase and may inhibit or slow down the galactose oxidase reaction. Accordingly, it is beneficial to maintain the hydrogen peroxide concentration in the reaction medium as low as possible. Surprisingly, it has been found that adding a catalase to a composition containing galactose oxidase, polysaccharide, and a one electron oxidant such as, for example, horseradish peroxidase, increases the substrate conversion.

Most one electron oxidants such as peroxidase, which may also be present in the system, are unable to fulfill this task as efficiently as a hydrogen peroxide remover such as catalase. Further, the hydrogen peroxide remover, such as catalase, typically cannot perform the function of the one electron oxidant, namely, activate the galactose oxidase from its inactive or semi form during the course of the reaction. Thus, the surprising combination of both a one electron oxidant and the hydrogen peroxide remover, when added to the galactose oxidase and the galactose oxidase substrate, are most able, in accordance with the present invention, to increase the activity level of galactose oxidase.

In accordance with the present invention, if catalase is selected as the hydrogen peroxide remover, catalase may be present in the system in a ratio greater than 0.1:1 with regards to galactose oxidase (Units catalase: IU galactose oxidase), more preferably greater than 1:1, even more preferably in a ratio greater than 5:1, and most preferably greater than 10:1.

Processes for drying and re-solubilizing oxidized carbohydrate gum compositions of the present invention are also within the scope of the present invention. Processes for drying and re-solubilizing oxidized carbohydrate gum, while described in brief hereinbelow, are the subject of a companion application filed on even date herewith Application No. ______(Attorney Docket No. V17050) “Process for the Production of Chemically or Enzymatically Modified Polysaccharides, and Products Made Thereby”, the disclosure of which is hereby incorporated by reference.

In fact, in accordance with another aspect of the present invention, a composition containing galactose oxidase substrate, galactose oxidase, one-electron oxidant and hydrogen peroxide remover may additionally include viscosity reducing agent. The addition of the viscosity reducing agent may improve the ability of the composition to be processed in industrial and full scale applications.

In another aspect of the present invention, some or all of the water and/or viscosity reducing agent, if present, may be removed from the composition. Thus, the concentrated or dry or solid compositions produced in accordance with the present invention may comprise polysaccharides, oxidized or unoxidized, or polysaccharides derivatives, oxidized or unoxidized, with or without viscosity reducing agent. Of course, other materials may be contained in the solid compositions as well.

The solid composition may be further processed, depending on its ultimate application. Preferably, the solid composition is milled through a sieve. Preferably the sieve has a size cutoff of greater than 0.05 mm, more preferably greater than 0.1 mm, and most preferably greater than 0.1 5 mm. Preferably the milling sieve has a size cutoff of less than 0.8 mm, more preferably less than 0.5 mm, and most preferably less than 0.3 mm. The range of size of the milling sieve is preferably from about 0.8 mm to about 0.05 mm, more preferably from about 0.5 mm to about 0.1 mm, and most preferably from about 0.1 5 mm to about 0.3 mm.

The solid compositions of the present invention are advantageous in exhibiting a stability which is superior to known aqueous compositions of oxidized carbohydrate gum. In particular, a solid composition of the present invention may be stored at room temperature without the addition of preservatives.

When re-solubilizing the oxidized carbohydrate gum, it is important to maintain all, or substantially all, of the aldehyde content of the dry product. The processes of the present invention minimize the loss of aldehyde content in an oxidized carbohydrate gum. Preferably, the re-solubilized oxidized carbohydrate gum includes at least approximately 70% of the original aldehyde content. More preferably, the re-solubilized gum includes approximately at least 80% of the original aldehyde content. Even more preferably, the re-solubilized oxidized gum includes approximately at least 90-100% of the original aldehyde content.

Re-solubilizing the compositions of the present invention involves at least adding a solvent (e.g., water) to the dried oxidized carbohydrate gum composition with the resulting composition being at a low pH. Moreover, as will be discussed below, the composition can be subjected to elevated temperatures and/or shear to enhance the resolubilization process. For example, elevating the temperature and/or using high shear while maintaining a low pH can assist in maintaining all, or substantially all, of the aldehyde content of the oxidized carbohydrate gum.

For example, it may be particularly advantageous, in accordance with the present invention, to utilize all of the following four elements in re-solubilizing an oxidized carbohydrate gum composition: 1) solvent (e.g., water), 2) low pH, 3) elevated temperature, and 4) shear. If these four elements are used together, they-may be performed in any order, but are preferably performed as 1 then 2 then 3 and 4 together. That is, preferably, water is first added to the mixture, the pH of the mixture is then adjusted, and then the mixture is simultaneously subjected to heating and shearing. Each element is described in more detail hereinafter.

Utilizing all four of the above listed elements allows the re-solubilization process to occur in substantially less time than without the use of the four elements. Specifically, the use of an elevated temperature, while maintaining the proper pH of the solution, allows the re-solubilization process to occur faster than at room temperature. Further, the use of shear, preferably high shear, allows the re-solubilization process to occur at a faster rate.

It appears that, when pH, temperature, and mixing time, are considered, the optimum conditions for dissolving cationic oxidized guar are: 1) dissolve the oxidized guar in acidified water such as acidified tap water, such that the resulting pH is about 5.4; 2) high shear, such as using an intensive turbulence blender (Waring Blender) at an elevated temperature, such as 90° C., and mixing for a period of time, such as 10 minutes.

Without further elaboration, it is believed that one skilled in the art can, using the preceding description, utilize the present invention to its fullest extent.

The following specific embodiments are, therefore, to be construed as merely illustrative, and not limitative of the remainder of the disclosure in any way whatsoever.

### EXAMPLES

### Example 1

### Determining the Amount of Galactose-6-aldehyde in Oxidized Raffinose and Oxidized Guar

Example 1 teaches a method to determine the amount of galactose 6-aldehyde in enzymatically oxidized guar.

The amount of galactose 6-aldehyde in oxidized raffinose or oxidized guar was determined according to the following procedure. Oxidized raffinose or oxidized guar samples were reduced by sodium borodeuteride treatment, hydrolyzed and reduced with sodium borodeuteride for a second time to form alditols. Acetylated alditols of mannose and galactose were baseline separated by gas chromatography (GC). The alditols of galactose and galactose 6-aldehyde elute at the same retention time. Using gas chromatography -mass spectrometry (GC-MS), the two galactitols could be distinguished because the incorporation of deuterium was different. Reduced galactose contained one deuterium (D1) and reduced galactose 6-aldehyde contained two deuteria (D2). Taking into account the isotope effects and the efficiency of labeling of non oxidized galactose the ratio of Dl :D2 in the sample was calculated with the masses 187:188, 217:218, and 289:290, which is a measure for the aldehyde percentage. The isotope effect was calculated from guar reduced by NaBH₄. The efficiency of guar labeling was determined by reduction of guar with NABD Method:

50 Fl of 110 mM raffinose and 50 Fl of 110 mM oxidized raffinose were labeled with deuterium using sodium borodeuteride (250 Fl 10 mg/ml NaBD₄ in 2M NH3, room temperature, 16 hours) followed by hydrolysis (0.5 ml trifluoroacetic acid, 1h at 121° C.) and a second NaBD₄ reduction (250 Fl 10 mg/ml NaBD₄ in 2M NH₃, 1h at 30° C.). The residues were derivatized by acetylation (3 ml acetic anhydride, 0.45 ml methylimidazole, 30 minutes at 30° C.) and analyzed by GC-MS (HP5890 GC, HP5972 series MSD with El fragmentation) equipped with a DB-1 column (60 m×0.25 I.D.×0.25 Fm film thickness, 70-280° C. with 4° C./min, 280° C. for 5 min) with splitless injection (splitless time 60 sec). 200 Fl 0.3% oxidized guar were analyzed as described for raffinose.

### Example 2

### Effect of Galactose Oxidase, Soybean Peroxidase and catalase on the aldehyde production using guar as a substrate

Enzyme activities expressed in Units or International Units as used in this and subsequent examples are defined as:

1. Galactose Oxidase [EC 1.1.3.9]: One International Unit (IU) will convert one micromol galactose per minute at pH 7 and 25 deg C.

2. Peroxidase [C 1.11.1.7]: One Unit will form 1.0 mg of purpurogallin from pyrogallol in 20 sec at pH 6.0 at 20 deg C.

3. Laccase [EC 1.10.3.2]: One U will produce a difference in absorption at a wavelength of 530 nm of 0.001/min at pH 6:5 at 30 deg C in a 3 ml reaction volume using syringaldazine as substrate.

4. Catalase [EC 1.11.1.6]: One Unit will decompose 1 micromol hydrogen peroxide per min at pH 7 at 25 deg C.

An experiment was performed to demonstrate the effect of adding both soybean peroxidase and catalase to a solution that included galactose oxidase and guar. Dry cationic guar (at 1% weight/volume) (Supercol U; Hercules Inc., Wilnington, Del.) was added to a beaker that contained 50 mM of Kpi (potassium phosphate) buffer at pH 7 with 0.5 mM CuSO₄, while stirring the buffer solution with a mechanical stirrer. The guar solution was divided over 8 Erlenmeyer flasks and then a mixture of catalase (Terminox, Novo Nordisk, Denmark, 50,000 U/ml) and/or soybean peroxidase (Soybean Peroxidase, Wiley Organics, 475 U/ml) and/or galactose oxidase (20 IU/ml, isolated from Dactylium dendroides fermentation, essentially as described by Tressel and Kossman, “A simple purification procedure for galactose oxidase”, Analytical Biochemistry Vol. 105, 150-153 (1980) was then added to the Erlenmeyer flasks, as indicated below in Table 16. When both galactose oxidase and soybean peroxidase were added to the flask, the galactose oxidase was pre-incubated with the soybean peroxidase for 1 minute at ambient temperature in a plastic tube. The guar solution was shaken in an incubator at room temperature at 260 rpm. After five (5) hours, the reaction was stopped by heating the mixture at 80° C. for ten (10) minutes. The wet samples were submitted for aldehyde analysis as described in example 1. The analytical results are indicated in Table 1 below.

As Table 1 shows, when both galactose oxidase and soybean peroxidase are present with the catalase, the highest level of aldehyde production is achieved.

TABLE 1

| Galactose Oxidase | Soybean Peroxidase | Terminox | Aldehyde |
|---|---|---|---|
| (50 IU/g guar) | (100 U/g guar) | (1500 U/g guar) | (%) |
| + | + | + | 27 |
| − | + | + | 0 |
| − | − | + | 0 |
| + | + | − | 13 |
| − | + | − | 0 |
| + | − | − | 3 |
| − | − | − | 0 |

### Example 3

### Conversion of guar to oxidized guar in presence of different activating agents

As shown in Table 2 and Table 3, the addition of one electron oxidants like ferricyanide and horseradish peroxidase to galactose oxidase prior to the reaction with guar leads to a significant increase in guar conversion as measured by the NaBD₄ reduction method as set forth in example 1. As discussed above, a large excess of ferricyanide is necessary to achieve an effective activation, whereas horseradish peroxidase may advantageously be added in catalytic amounts.

A series of Erlenmeyer flasks (250 ml) containing 20 ml 0.3% neutral guar (Supercol U, Hercules, Wilmington, Del.) in 50 mM KPi 7.0 buffer, 0.5 mM CuSO₄ and 1.6×10⁶ Units catalase per g guar (Boehringer; beef liver; 2.6×10⁵ U/ml) was incubated at 5° C. for approximately 30 minutes. The solutions were subsequently supplemented with 200 Fl of pre-incubated galactose oxidase sample containing the amounts of ferricyanide or horseradish peroxidase quoted in the tables (final galactose oxidase concentration was 150 International Units per gram (IU/g) guar. After enzyme addition the reaction mixtures were incubated under vigorous shaking (rotary shaker with speed at 300 rpm). The enzymatic reaction was stopped by heating the samples in a waterbath for 10 minutes at 80° C. Final levels of oxidation in the different samples were determined using a NaBD₄ reduction method.

TABLE 2
Activation of Galactose oxidase with Ferricyanide

| [Fe³⁺] | Fe:GaOx ratio | | Aldehyde Production |
|---|---|---|---|
| (nmol) | (nmol:nmol) | Oxidation (%) | (Fmol/IU) |
| 10000 | 113636 | 81 | 12 |
| 1000 | 11364 | 84 | 12.4 |
| 100 | 1136 | 21 | 3.2 |
| 10 | 114 | 13 | 2 |
| 1 | 11 | 14 | 2 |

### Example 4

### Influence of the Ratio of Galactose Oxidase and Soybean Peroxidase on Galactose Oxidase activity under Assay Conditions

A. Description of the measurement principle

Because peroxidases display the capability to activate galactose oxidase, a peroxidase free assay system was selected to follow galactose oxidase activity. In this way, the active galactose oxidase fraction and the inactive fraction of all galactose oxidase activity present can be determined. For this purpose, oxygen consumption was monitored as a measure of galactose oxidase activity in a biological oxygen monitor (BOM). Galactose oxidase activity is expressed as a percentage (%) of oxygen consumption per minute per ml galactose oxidase sample.

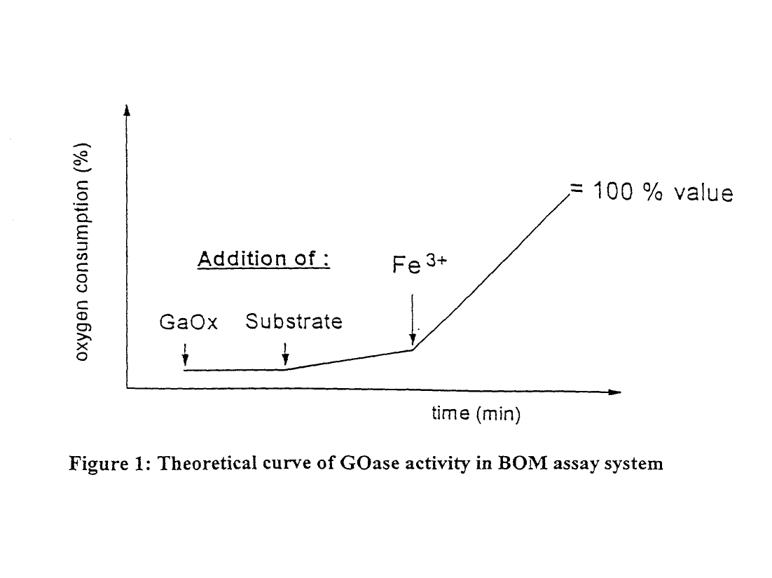

Addition of 1 mM ferricyanide is assumed to result in maximum activation of all enzyme present in the BOM compartment. The rate of oxygen consumption measured after ferricyanide addition is considered to be the substantially 100% value and all determined activities are correlated to this value. The principle of the measurement is further illustrated in FIG. 1. In this way the partially active galactose oxidase fraction (and inactive fraction) from a sample can be determined.

B. Determination of the optimum Soybean peroxidase:Galactose oxidase ratio.

To determine whether an optimal soybean peroxidase concentration for galactose oxidase activation exists, galactose oxidase (8 IU/mg solid, Sigma) was incubated with increasing levels of soybean peroxidase (Soybean Peroxidase, Wiley Organics, 8750 U/g solids). The activating effect of the applied ratio was determined by measuring the galactose oxidase activity (oxygen consumption) with a Biological Oxygen Monitor (BOM) (model 5300; YSI Incorporated, Yellow Springs, Ohio) connected to an Oxygen probe (model 5331; YSI Incorporated, Yellow Springs, Ohio). For each assay, a sample chamber was filled with 5 ml BOM assay mixture containing 50 mM KPi buffer pH 7.0, 200 mM D-galactose and 0.5 mM CuSO₄. This solution was vigorously stirred for several minutes until complete air-O₂ saturation was obtained. To prevent inhibition due to H₂O₂ formation during the reaction, 50 Fl catalase (from beef liver; 260.000 U/ml; Boehringer) was added to the reaction chamber. The oxygen probe was inserted into the reaction chamber and, after signal stabilization, 10 Fl of soybean peroxidase/galactose oxidase sample (see Table 4) was added.

Each soybean peroxidase/galactose oxidase sample mixture was freshly prepared and subsequently pre-incubated for 5 min at room temperature, before it was added to the reaction chamber. After addition, the oxygen consumption was monitored in time. After about 5 minutes, 50 Fl 50 mM Fe³⁺ solution (K₃Fe(CN)₆ in 50 mM KPi pH 7.0) was added to determine the maximum activity. This equaled the substantially 100% value.

To prepare a range of samples with an increasing soybean peroxidase:galactose oxidase ratio, a soybean peroxidase solution of 400 U/ml (in KPi buffer) and a galactose oxidase (Sigma; 8 IU/mg solid) solution of 100 IU/ml was used. Different volumes of soybean peroxidase, galactose oxidase and buffer (see Table 4) were dispensed and mixed in a 1 ml eppendorf tube.

TABLE 4
Dilution Series of Soybean Peroxidase
and Galactose Oxidase to Obtain an
Increasing Soybean Peroxidase:Galactose Oxidase Ratio.

| | | | | Amount | |
|---|---|---|---|---|---|
| | | | | (10 μl volume) | |
| | | | | added to 5 mL BOM | |
| | | | | mixture | |
| SBP: | | 100 IU/ml | | | Galactose |
| GaOx | 400 U/ml | galactose | KPi 50 mM | | oxidase |
| Ratio | SBP (μl) | oxidase (μl) | pH 7.0 (μl) | SBP (U) | (IU) |
| without | 0 | 100 | 100 | 0 | 0.5 |
| SBP | | | | | |
| 0.01 | 2.5 (10*) | 100 | 97.5 | 0.005 | 0.5 |
| 0.02 | 5.0 (10*) | 100 | 95 | 0.01 | 0.5 |
| 0.1 | 2.5 | 100 | 97.5 | 0.05 | 0.5 |
| 0.2 | 5.0 | 100 | 95 | 0.1 | 0.5 |
| 0.5 | 12.5 | 100 | 87.5 | 0.25 | 0.5 |
| 1 | 25 | 100 | 75 | 0.5 | 0.5 |
| 2 | 50 | 100 | 50 | 1.0 | 0.5 |
| 4 | 100 | 100 | 0 | 2.0 | 0.5 |

TABLE 5
Galactose Oxidase activity in presence of various amounts of
Soy Bean Peroxidase as measured with a BOM

| Soy Bean Peroxidase:Galactose Oxidase | |
|---|---|
| Ratio (U:IU) | Relative activity (%) |
| No Soy Bean Peroxidase | 15 |
| 0.01 | 37 |
| 0.02 | 82 |
| 0.1 | 105 |
| 0.2 | 105 |
| 0.5 | 109 |
| 1 | 92 |
| 2 | 89 |
| 4 | 97 |

### Example 5

### Galactose oxidase:Soybean peroxidase ratio under preparative conditions

To determine the addition level of soybean peroxidase to activate galactose oxidase, increasing soybean peroxidase:galactose oxidase ratios were tested for aldehyde production using neutral guar as substrate.

A series of 9 Erlenmeyer flasks (250 ml) containing 20 ml 0.3% neutral guar (Supercol U, Hercules, Wilmington, Del.) in 50 mM KPi pH 7.0 buffer, 0.5 mM CuSO₄ and 1.6×10⁶ Units catalase per gram of guar (Boehringer; beef liver; 2.6×10⁵ U/ml) were incubated at about 5° C. for approximately 30 minutes. The solutions were subsequently supplemented with 1 ml of pre-incubated soybean peroxidase:galactose oxidase sample containing an increasing soybean peroxidase: galactose oxidase ratio for 15 min at ambient temperature (see Table 5, final galactose oxidase concentration was 150 IU/g guar). After adding the enzyme, the reaction mixtures were incubated over night under vigorous shaking (Rotary shaker with speed at 300 rpm). The enzymatic reaction was stopped by heating the samples in a waterbath for 10 minutes at about 80° C. Final levels of oxidation in the different samples are determined using the NaBD₄ reduction method as described in Example 1

TABLE 6
Effect of increasing Soy Bean Peroxidase Galactose Oxidase Ratio
on yield of oxidized guar as expressed in % aldehyde produced

| | | Galactose | | |
|---|---|---|---|---|
| | SBP | Oxidase | SBP:Galactose | |
| SBP Dilution | Solution | Solution (18 | Oxidase Ratio | |
| Range (U/ml) | (ml) | IU/ml)(ml) | (U/IU) | % aldehyde |
| 0 | 0.5 | 0.5 | 0 | 0 |
| 0.18 | 0.5 | 0.5 | 0.01 | 1 |
| 0.36 | 0.5 | 0.5 | 0.02 | 1 |
| 1.8 | 0.5 | 0.5 | 0.1 | 3 |
| 3.6 | 0.5 | 0.5 | 0.2 | 5 |
| 9.0 | 0.5 | 0.5 | 0.5 | 48 |
| 18 | 0.5 | 0.5 | 1 | 51 |
| 36 | 0.5 | 0.5 | 2 | 50 |
| 72 | 0.5 | 0.5 | 4 | 44 |

One half (0.5) ml of a 18 IU/ml galactose oxidase solution (Sigma; 8 IU/mg) was mixed with 0.5 ml of the above described soybean peroxidase solutions (soybean peroxidase with 8.75 U/mg solid) and pre-incubated for 15 minutes at room temperature.

### Example 6

### Determination of Galactose Oxidase:Horseradish Peroxidase Ratio

To determine the level of horseradish peroxidase to activate galactose oxidase, increasing horseradish peroxidase:galactose oxidase ratios were tested for aldehyde production using neutral guar as substrate. Incubations were performed at either room temperature or at 5° C.

Pre-incubation of galactose oxidase with increasing levels of horseradish peroxidase results in a clear increase in activity level and thereof yielding in an elevated oxidation level (see Table 4).

Two series of 9 Erlenmeyer flasks (250 ml), each containing 20 ml 0.3% Neutral guar (Supercol U, Hercules, Wilmington, Delaware) in 50 mM KPi pH 7.0 buffer and 1.6.×10⁶ Units catalase per gram guar (Boehringer; beef liver; 2.6×10⁵ U/ml) were either incubated at room temperature or at 5° C. for approximately 30 minutes. Subsequently both series were supplemented with 1 ml of pre-incubated horseradish peroxidase:galactose oxidase sample containing an increasing horseradish peroxidase:galactose oxidase ratio (see Table 6; final galactose oxidase concentration was 150 IU/g guar). Horse radish peroxidase was obtained from Sigma (200 U/mg). After enzyme addition, the reaction mixtures were incubated under vigorous shaking (Rotary shaker with speed at 300 rpm). The enzymatic reaction was stopped by heating the samples in a waterbath for 10 minutes at 80° C. Final levels of oxidation in the different samples were determined using an iodometric titration method. The iodometric assay for aldehyde (I₂+CHOv COOH+2I⁻) uses titration of excess I₂ with sodium thiosulfate.

TABLE 7
Dilution Series of Horseradish Peroxidase
and Galactose Oxidase to Obtain an
Increasing Horseradish Peroxidase:Galactose Oxidase Ratio

| | Dilution Series | | | | |
|---|---|---|---|---|---|
| | | Kpi | | | |
| HRP | Volume | Buffer | Dilution | | |
| Stock | HRP | 50 mM | Range | HRP Activity in | HRP:Galactose |
| Solution | Stock | pH | HRP | 0.5 ml Solution | oxidase |
| (U/ml) | (ml) | 7.0 (ml) | (U/ml) | (U) | Ratio |
| 400 | 1.0 | 0 | 400 | 200 | 22.2 |
| 400 | 0.2 | 1.8 | 40 | 20 | 2.2 |
| 40 | 1.0 | 1.0 | 20 | 10 | 1.12 |
| 20 | 1.0 | 1.0 | 10 | 5 | 0.56 |
| 10 | 0.8 | 1.2 | 4 | 2 | 0.22 |
| 4 | 1.0 | 1.0 | 2 | 1 | 0.112 |
| 2 | 1.0 | 1.0 | 1 | 0.5 | 0.056 |
| 1 | 0.4 | 1.6 | 0.2 | 0.1 | 0.012 |
| 1.2 | 1.0 | 1.0 | 0.1 | 0.05 | 0.006 |

TABLE 8
Effect of increasing Horse Radish Peroxidase Galactose Oxidase Ratio
on yield of oxidized guar as expressed in % aldehyde produced
at different temperatures

| Horse Radish | % Aldehyde | |
|---|---|---|
| Peroxidase: | produced at room | |
| Galactose Oxidas | temperature (by | % Aldehyde produced at 5° C. |
| (U:IU) | titration method)* | (by titration method)* |
| 0.006 | 43 | 29 |
| 0.012 | 28 | 27 |
| 0.056 | 35 | 27 |
| 0.112 | 48 | 25 |
| 0.22 | 41 | 34 |
| 0.56 | 73 | 136 |
| 1.12 | 92 | 143 |
| 2.2 | 76 | 146 |
| 22.2 | 82 | 141 |

*Please note that the titration method is an unspecific method to measure aldehyde content of oxidized guar. Under the analytical conditions, decomposition of the oxidized product can occur, leading to formation of additional aldehyde groups. This leads to values exceeding the theoretical 100%. Only relative comparison of the numbers is therefore possible.

### Example 7

### Determination Of The Optimum Amount Of Catalase In The Production Of Cationic Oxidized Guar

20 ml aliquots of a 0.6% cationic guar solution (N-RANCE 3198; Hercules) in 50 mM phosphate buffered medium (pH 7.0) were measured into 250 ml shaking flasks. To the cationic guar solutions, catalase (Reyonet S ex Nagase, Japan, 50.000 CtUN/g) was added in various amounts (see table 8). After adding catalase, a mixture of soybean peroxidase (Organic Technologies, Coshocton, Ohio) and galactose oxidase (From Dactylium dendroides fermentation) was added to account for a final enzyme concentration of 50 IU galactose oxidase per gram of guar and 50 U/g soybean Peroxidase. The galactose oxidase—soybean peroxidase mixture was preincubated 15 minutes before adding the same to the guar solutions. The shaking flasks were then incubated for 5 hours at room temperature on a rotary shaker.

The conversion of the cationic guar to oxidized cationic guar was measured by the NaBD₄ reduction method as described in example 1. The results are expressed as percent (%) of galactose units converted to aldehyde. The experiment was performed twice and the results are summarized in Table 9.

| Reyonet S | Reyonet S | Oxidation average |
|---|---|---|
| (U/IU Galactose oxidase) | (Units of catalase/g guar) | (% aldehyde) |
| 0 | 0 | 13.5 |
| 1 | 50 | 19 |
| 2 | 100 | 20.5 |
| 5 | 250 | 23 |
| 10 | 500 | 2515 |
| 20 | 1000 | 25.5 |

As illustrated in Table 9, the results of this experiment show a significant increase in substrate conversion upon addition of a sufficient amount of catalase as a third enzyme to a reaction mixture containing galactose oxidase and a peroxidase.

### Example 8

### Amount of Catalase To Achieve Improved Conversion of Guar

The experiment described in Example 7 above was repeated with a different catalase (Terminox 50L ex Novo Nordisk, 55.000 U/ml). Enzyme dosages used in this experiment were 58 IU/g galactose oxidase, 116 U/g Horseradish Peroxidase (Sigma; 200 U/mg) and varying amounts of Terminox SOL as stated in table 10. The other experimental conditions were the same as described for Example 7. Substrate conversion in relation to catalase concentration is shown below in Table 10.

TABLE 10
Influence of Terminox 50L concentration on guar conversion

| Terminox 50L | Terminox 50L | Oxidation average |
|---|---|---|
| (U/IU Galactose oxidase) | (U/g guar) | (% aldehyde) |
| 0 | 0 | 20 |
| 10.4 | 605 | 33 |
| 20.6 | 1210 | 34.5 |
| 103 | 6050 | 34.5 |
| 206 | 12100 | 33.5 |
| 309 | 18104 | 34 |

Again, the conversion of cationic guar to oxidized cationic guar increased significantly upon addition of catalase. In this case, the lowest catalase concentration of 605 U/g of guar was sufficient to achieve the conversion increase.

As Example 7, the outcome of this experiment shows a significant increase in substrate conversion upon addition of catalase to a mixture containing galactose oxidase and a peroxidase.

### Example 9

### Amount of Peroxidase Needed to Achieve Improved Conversion of Guar As Substrate

To illustrate the influence of a peroxidase on the efficiency of galactose oxidase, a series of experiments is performed with varying amounts of peroxidase in the reaction medium. The concentrations of galactose oxidase (from Dactylium dendroides fermentation) and catalase (Boehringer Mannheim, 260,000 U/ml) are maintained at constant levels and the conversion of the substrate was monitored as described in the examples above. In this Example, soybean peroxidase (Organic Technologies, Coshocton, Ohio, 475 U/ml) is used.

The galactose oxidase dosage is 50 IU/g of guar and the catalase (2800 U/mg) is used at 10,000 U/g of guar. Other experimental conditions are substantially the same as described in Example 8. Soybean peroxidase dosages and conversion results are summarized below in Table 11.

| SBP | SBP | Oxidation average (% |
|---|---|---|
| (U/IU Galactose oxidase) | (U/g guar) | aldehyde) |
| 0 | 0 | 1.5 |
| 0.1 | 5 | 31 |
| 0.2 | 10 | 34 |
| 0.5 | 25 | 35.5 |
| 1 | 50 | 39 |
| 2 | 100 | 39.5 |

From Table 11, it can be seen that the influence of the Peroxidase is more pronounced than that of the catalases studied in Examples 7 and 8. Without peroxidase present in the reaction medium, a conversion of only 1.5% was achieved under the given reaction conditions. On the other hand, conversion immediately increased to approximately 31% upon addition of the lowest dosage of soybean peroxidase.

### Example 10

### Amount of a Laccase Necessary to Achieve Improved conversion of guar as substrate

In this example, Laccase (ex Novo Nordisk, 1000 LAMU/g) was used as the Peroxidase. Galactose oxidase dosage was 50 IU/g of guar, catalase (260,000 U/ml, Boehringer Mannheim) was used at 10.000 U/g of guar. Other experimental conditions were the same as described in example 1. Laccase dosages and conversion results are summarized in Table 12.

TABLE 12
Influence of Laccase concentration on guar conversion

| Laccase | | Oxidation average (% |
|---|---|---|
| (U/IU Galactose oxidase | Laccase (U/g guar) | aldehyde) |
| 0 | 0 | 1.5 |
| 0.1 | 5 | 13.5 |
| 0.2 | 10 | 15.5 |
| 0.5 | 25 | 21 |
| 1 | 50 | 26 |
| 2 | 100 | 26.5 |

Table 12 shows that the influence of the laccase is somewhat less pronounced than the effect observed for soybean peroxidase. However, it appears that the enzyme still significantly increases product conversion, even at the lower dosage levels when compared to the sample oxidized in the absence.

### Example 11

### Effect of Polyethylene Glycol on Guar Oxidation

Example 11 demonstrates that 1% guar added to 5% polyethylene glycol 20,000 can be efficiently converted to the polyaldehyde derivative.0.2 gram neutral Guar gum (Supercol U; Hercules Incorporated, Wilmington, Delaware) was added to a 50 ml plastic tube containing 20 ml of 50 mM potassium phosphate buffer, pH 7.0, supplemented with 0.5 mM CuSO₄ and 5% polyethylene glycol 20,000. After thoroughly mixing, this solution was transferred to a 250 ml Erlenmeyer flask and 200 μl 260.000 ^{IU}/ₘₗ catalase (beef liver, Boehringer Mannheim) was added. Prior to the enzyme reaction, the guar/polyethylene glycol solution was shaken in a rotary shaker (300 rpm; ambient temperature) to ensure air saturation of the solution.

30 IU galactose oxidase was pre-incubated with 60 U of horseradish peroxidase (200 units/mg, Sigma) for approximately 15 min. at ambient temperature. After the pre-incubation period, the galactose oxidase/HRP mixture was added to the guar/polyethylene glycol solution. This reaction mixture was incubated on a rotary shaker (300 rpm) for 22 hours at ambient temperature. After 22 hours incubation the reaction was stopped by heating the 20 ml reaction mixture for 10 min. at 80° C. in a water bath. The formed aldehyde level was determined using a NABD₄ reduction method, described below in Example 1. 63% of all the galactose residues originally present in the sample had been converted into their 6-aldehyde derivative.

### Example 12

### Effect of Polyethylene Glycol on Guar Oxidation

Example 12 demonstrates that the galactose of guar can efficiently be converted to the aldehyde, in a mixture containing 1% guar to which 5% dry polyethylene glycol 20.000 was added.0.2 gram dry Supercol U guar was added to a 50 ml plastic tube containing 20 ml potassium phosphate buffer, 50 mM, pH 7.0, supplemented with 0.5 mM CuSO₄. This suspension was thoroughly mixed until the guar was completely hydrated-and dissolved. Subsequently 1.0 g polyethylene glycol 20,000 was added and dissolved-into the guar solution. The guar/polyethylene glycol solution was transferred to a 250 ml Erlenmeyer flask and 200 μl 260.000 ^{IU}/ₘₗ catalase (beef liver, Boehringer Mannheim) was added. Prior to the enzyme reaction, the guar/polyethylene glycol solution was shaken in a rotary shaker (300 rpm; ambient temperature) to ensure air saturation of the solution. 30 IU galactose oxidase activity was pre-incubated with 60 units of horseradish peroxidase (200 units/mg, Sigma) for approximately 15 min. at ambient temperature. After the pre-incubation period, the galactose oxidase/HRP mixture was added to the guar/polyethylene glycol solution. This reaction mixture was incubated on a rotary shaker (300 rpm) for 22 hours at ambient temperature. After 22 hours incubation the reaction was stopped by heating the 20 ml reaction mixture for 10 min. at 80° C. in a water bath. The formed aldehyde level was determined using a NaBD₄ reduction method, described above in Example 1. 95% of all the galactose residues, originally present in the neutral guar gum sample, had been converted into their 6-aldehyde derivative.

### Example 13

### Efficiency of Enzymatic Oxidation of Guar and Raffinose at Different Polyethylene Glycol Concentrations

Example 13 demonstrates the efficiency of the enzymatic oxidation of guar in guar/polyethylene glycol mixtures of different concentrations.

Example 13 includes a number of guar and raffinose oxidations performed under various reaction conditions following the standard procedure described in Examples 11 and 12. The hydration method specifies the order of addition of polyethylene glycol and guar to the water phase, G6P representing addition of dry guar to an aqueous polyethylene glycol solution and P6G representing addition of dry polyethylene glycol to an aqueous guar paste. Aldehyde contents of the guars were measured by the NaBD₄ reduction method. Enzyme productivity was defined as amount of aldehyde produced [Fmol] per IU of galactose oxidase.

TABLE 13
Examples for oxidation of Supercol U and Raffinose in polyethylene glycol mixtures

| | polyethylene | | | | GOase: | | | incubation | |
|---|---|---|---|---|---|---|---|---|---|
| Guar | glycol 20.000 | hydration | GOase | catalase | HRP | Aldehyde | productivity | time | Temperature |
| (%) | (%) | method | (IU/g) | (U/g) | (IU/U) | (%) | (3 mol/IU) | (h) | (° C.) |
| 0.3 | 0 | G -> P | 150 | 1600000 | 1:2 | 38 | 5.6 | 20 | 6 |
| 0.3 | 1 | | 150 | 1600000 | 1:2 | 65 | 9.6 | 20 | 6 |
| 0.3 | 2 | | 150 | 1600000 | 1:2 | 75 | 11.2 | 20 | 6 |
| 0.3 | 5 | | 150 | 1600000 | 1:2 | 52 | 7.8 | 20 | 6 |
| 1 | 1 | | 45 | 240000 | 1:2 | 37 | 18.2 | 20 | 6 |
| 2 | 2 | | 22.5 | 1600000 | 1:2 | 22 | 21.8 | 20 | 6 |
| 1 | 1 | | 150 | 480000 | 1:2 | 66 | 9.8 | 20 | 6 |
| 2 | 2 | | 150 | 240000 | 1:2 | 58 | 8.6 | 20 | 6 |
| 1 | 2 | G -> P | 150 | 240000 | 1:2 | 65 | 9.6 | 22 | 6 |
| 2 | 2 | | 150 | 120000 | 1:2 | 57 | 8.4 | 22 | 6 |
| 2 | 2 | | 24 | 120000 | 1:2 | 22 | 20.4 | 22 | 6 |
| 3 | 2 | | 150 | 80000 | 1:2 | 49 | 7.2 | 22 | 6 |
| 1 | 3 | | 150 | 240000 | 1:2 | 59 | 8.8 | 22 | 6 |
| 2 | 3 | | 150 | 120000 | 1:2 | 49 | 7.2 | 22 | 6 |
| 3 | 3 | | 150 | 80000 | 1:2 | 45 | 6.6 | 22 | 6 |
| 4 | 3 | | 150 | 60000 | 1:2 | 37 | 5.4 | 22 | 6 |
| 3 | 5 | | 150 | 80000 | 1:2 | 31 | 4.6 | 22 | 6 |
| 4 | 5 | | 150 | 60000 | 1:2 | 27 | 4 | 22 | 6 |
| 5 | 5 | | 150 | 48000 | 1:2 | 22 | 3.2 | 22 | 6 |
| 0.3 | 3 | G -> P | 150 | 720000 | 1:3 | 57 | 8.4 | 3 | 22 |
| 0.6 | 3 | | 75 | 360000 | 1:3 | 39 | 11.6 | 3 | 22 |
| 1 | 3 | | 45 | 217000 | 1:3 | 25 | 12.4 | 3 | 22 |
| 2 | 3 | | 22.5 | 108000 | 1:3 | 12 | 11.8 | 3 | 22 |
| 3 | 3 | | 15 | 72000 | 1:3 | 9 | 13.4 | 3 | 22 |
| 4 | 3 | | 11 | 54000 | 1:3 | 4 | 8 | 3 | 22 |
| 0.3 | 3 | G -> P | 150 | 720000 | 1:3 | 86 | 12.8 | 6 | 22 |
| 0.6 | 3 | | 75 | 360000 | 1:3 | 69 | 20.4 | 6 | 22 |
| 1 | 3 | | 45 | 217000 | 1:3 | 54 | 26.6 | 6 | 22 |
| 2 | 3 | | 22.5 | 108000 | 1:3 | 27 | 26.6 | 6 | 22 |
| 3 | 3 | | 15 | 72000 | 1:3 | 16 | 23.8 | 6 | 22 |
| 4 | 3 | | 11 | 54000 | 1:3 | 12 | 23.8 | 6 | 22 |
| 0.3 | 3 | G -> P | 150 | 720000 | 1:3 | 86 | 12.8 | 24 | 22 |
| 0.6 | 3 | | 75 | 360000 | 1:3 | 71 | 21 | 24 | 22 |
| 1 | 3 | | 45 | 217000 | 1:3 | 61 | 30.2 | 24 | 22 |
| 2 | 3 | | 22.5 | 108000 | 1:3 | 26 | 25.6 | 24 | 22 |
| 3 | 3 | | 15 | 72000 | 1:3 | 16 | 23.8 | 24 | 22 |
| 4 | 3 | | 11 | 54000 | 1:3 | 10 | 19.8 | 24 | 22 |
| 1 | 1 | G -> P | 150 | 260000 | 1:2 | 47 | 7 | 22 | 22 |
| 1 | 2 | | 150 | 260000 | 1:2 | 78 | 11.6 | 22 | 22 |
| 1 | 5 | | 150 | 260000 | 1:2 | 63 | 9.4 | 22 | 22 |
| 1 | 10 | | 150 | 260000 | 1:2 | 7 | 1 | 22 | 22 |
| 1 | 30 | | 150 | 260000 | 1:2 | 1 | 0.2 | 22 | 22 |
| 20 ml | 1 | Raffinose | 150 | 260000 | 1:2 | 96 | 14.2 | 22 | 22 |
| 66 | | | | | | | | | |
| mM | | | | | | | | | |
| 20 ml | 2 | Raffinose | 150 | 260000 | 1:2 | 99 | 14.6 | 22 | 22 |
| 66 | | | | | | | | | |
| mM | | | | | | | | | |
| 20 ml | 5 | Raffinose | 150 | 260000 | 1:2 | 97 | 14.4 | 22 | 22 |
| 66 | | | | | | | | | |
| mM | | | | | | | | | |
| 20 ml | 10 | Raffinose | 150 | 260000 | 1:2 | 71 | 10.4 | 22 | 22 |
| 66 | | | | | | | | | |
| mM | | | | | | | | | |
| 20 ml | 30 | Raffinose | 150 | 260000 | 1:2 | 43 | 6.4 | 22 | 22 |
| 66 | | | | | | | | | |
| mM | | | | | | | | | |
| 1 | 1 | P -> G | 150 | 260000 | 1:2 | 95 | 14 | 22 | 22 |
| 1 | 3 | | 150 | 260000 | 1:2 | 96 | 14.2 | 22 | 22 |
| 1 | 5 | | 150 | 260000 | 1:2 | 95 | 14 | 22 | 22 |
| 1 | 6 | | 150 | 260000 | 1:2 | 88 | 13 | 22 | 22 |
| 1 | 7 | | 150 | 260000 | 1:2 | 85 | 12.6 | 22 | 22 |
| 1 | 8 | | 150 | 260000 | 1:2 | 72 | 10.6 | 22 | 22 |
| 1 | 9 | | 150 | 260000 | 1:2 | 58 | 8.6 | 22 | 22 |
| 1 | 10 | | 150 | 260000 | 1:2 | 41 | 6 | 22 | 22 |
| 1 | 15 | | 150 | 260000 | 1:2 | 9 | 1.4 | 22 | 22 |
| 1 | 20 | | 150 | 260000 | 1:2 | 7 | 1 | 22 | 22 |
| 4 | 2 | P -> G | 9.5 | 10000 | 1:2 | 9 | 21 | 2.5 | 22 |
| 4 | 2 | | 9.5 | 10000 | 1:2 | 13 | 30.4 | 24 | 22 |
| 4 | 2.5 | P -> G | 48 | 10000 | 1:2 | 34 | 16 | 3 | 22 |
| 4 | 2.5 | | 48 | 10000 | 1:2 | 36 | 16.8 | 6 | 22 |
| 4 | 2.5 | | 48 | 10000 | 1:2 | 42 | 19.6 | 20 | 22 |
| 8 | 5 | P -> G | 24 | 10000 | 1:2 | 11 | 10.2 | 1 | 35 |
| 8 | 5 | | 24 | 10000 | 1:2 | 11 | 10.2 | 2 | 35 |
| 8 | 5 | | 24 | 10000 | 1:2 | 13 | 12.2 | 4 | 35 |
| 8 | 5 | | 24 | 10000 | 1:2 | 14 | 13 | 22 | 35 |

### Example 14

### Preparation of oxidized cationic guar in presence of poyethylene glycol

In a 101 container 51 of a 50 mM potassium phosphate buffer solution with a pH of 7 was prepared. While the solution was stirred, 25mg CuSO₄ was added. 50 g PEG 6000 (BASF, Ludwigshafen, Germany) were added to the buffer solution which was stirred with a mechanical stirrer until the PEG was fully dissolved. 50 g cationic guar (N-Hance 3198, Hercules Incorporated, Wilmington, Del.) was then added to the solution, which was further stirred until the composition was homogeneous. The thus prepared mixture contained 1% wv cationic guar and 1% w/v PEG 6000. 1.5 ml of catalase (Reyonet S, Nagase, Japan, 50.000 U/ml) were added to the solution.

The guar mixture was then poured into a 7 l fermentor (Biocontroler ADI 1030, Applicon, Schiedam, Netherlands). The stirrer was adjusted to a speed of 1200 rpm, the solution was aerated with compressed air at a rate of 1.277 l/min. A mixture of 125 ml of a galactose oxidase preparation (20 IU/ml, from Dactylium dendroides fermentation) and 10.53 ml soy bean peroxidase solution (Wiley Organics; 475 U/ml) was prepared and incubated for 5 min, after which the mixture was added to the fermentor. The reaction mixture was aerated under maintained agitation for five hours at ambient temperature to allow the oxidation to proceed.

After 5 h reaction time, the content of the fermentor was poured slowly and under gentle stirring into a 101 container charged with 51 of isopropanol. The mixture was stirred for another two hours, the precipitated oxidized cationic guar was then allowed to settle overnight. The reaction product was recovered by filtration over a Whatman-1 filter paper using a Büchner funnel. The collected precipitate was washed twice with 1 l 50% isopropanol in water. The washed product was allowed to dry overnight at ambient temperature and pressure in the fume cupboard.

The dried product was milled on a Retsch DR100 mill with decreasing sieve sizes, starting from a size cutoff of 0.8 mm, down to a final size cutoff of 0.15 mm. Total solids of the dried and milled material was determined by placing a weighed sample in a vacuum oven at 30° C. for 16 h. Conversion was measured by the reduction method described in Example 1 and was found to be 38% in the dry product.

### Example 15

### Preparation of oxidized cationic guar in presence of poyethylene glycol

In a 250 ml beaker, 200 ml of a 50 mM potassium phosphate buffer solution with a pH of 7 was prepared and supplemented with 50 mM CuSO₄. 10 g PEG 6000 (BASF, Ludwigshafen, Germany) were added to the buffer solution which was stirred with a mechanical stirrer until the PEG was fully dissolved. 10 g cationic guar (N-Hance 3198, Hercules Inc., Wilmington, Del.) were then added to the solution, which was further stirred until the composition was homogeneous. The thus prepared mixture contained 5% wv cationic guar and 5% w/v PEG 6000. 60 ml of catalase (Reyonet S, Nagase, Japan, 50.000 U/ml) were added to the solution. A mixture of 75 ml of a galactose oxidase preparation (20 IU/ml, from Dactylium dendroides fermentation) and 6.32 ml soy bean peroxidase solution (Wiley Organics, 475 U/ml) was prepared and incubated for 5 min, after which the mixture was added to the reaction mixture. The reaction mixture was poured into a 1 l Erlenmeyer flask which was shaken for 5 h in an incubator at 300 rpm.

After 5 h reaction time, the content of the Erlenmeyer flask was poured slowly and under gentle stirring into a 1 l beaker charged with 200 ml of isopropanol. The mixture was stirred for another two hours, the precipitated oxidized cationic guar was then allowed to 20! settle overnight. The reaction product was recovered by filtration over a Whatman-1 filter paper using a Büchner fennel. The collected precipitate was washed twice with 50 ml 50% isopopanol in water. The washed product was allowed to dry overnight at ambient temperature and pressure in the fume cupboard.

The dried product was milled on a Retsch DR100 mill with decreasing sieve sizes, starting from a size cutoff of 0.8 mm, down to a final size cutoff of 0.15 mm. Total solids of the dried and milled material was determined by placing a weighed sample in a vacuum oven at 30° C. for 16 h. Conversion was measured by the reduction method described in example 10 and was found to be 30% in the dry product.

### Example 16

### Preparation of oxidized cationic guar in presence of poyethylene glycol

In a 250 ml beaker, 100 ml of a 50 mM potassium phosphate buffer solution with a pH of 7 was prepared and supplemented with 50 mM CuSO₄. 3.5 g PEG 6000 (BASF, Ludwigshafen, Germany) were added to the buffer solution which was stirred with a mechanical stirrer until the PEG was fully dissolved. 3.5 g cationic guar (N-Hance 3198, Hercules Inc., Wilmington, Del.) were then added to the solution, which was further stirred until the composition was homogeneous. The thus prepared mixture contained 3.5% wv cationic guar and 3.5% w/v PEG 6000. 15.75 ml of catalase (Reyonet S, Nagase, Japan, 50.000 U/ml) were added to the solution. A mixture of 19.7 ml of a galactose oxidase preparation (20 IU/ml, from Dactylium dendroides fermentation) and 1.66 ml soy bean peroxidase solution (Wiley Organics, 475 U/ml) was prepared and incubated for 5 min, after which the mixture was added to the reaction mixture. The reaction mixture was poured into a 500 ml Erlenmeyer flask which was shaken for 5 h in an incubator at 300 rpm.

After 5 h reaction time, the content of the Erlenmeyer flask was poured slowly and under gentle stirring into a 1 liter beaker charged with 100 ml of isopropanol. The mixture was stirred for another two hours, the precipitated oxidized cationic guar was then allowed to settle overnight. The reaction product was recovered by filtration over a Whatman-1 filter paper using a Büchner funnel. The collected precipitate was washed 4 times with 50 ml 50% isopopanol in water. The washed product was allowed to dry overnight at ambient temperature and pressure in the fume cupboard. The dried product was milled on a Retsch DR100 mill with decreasing sieve sizes, starting from a size cutoff of 0.8 mm, down to a final size cutoff of 0.15 mm. Total solids of the dried and milled material was determined by placing a weighed sample in a vacuum oven at 30° C. for 16 h. Conversion was measured by the reduction method described in Example 1 and was found to be 28% in the dry product.

### Example 17

### Application of the product from Examples 14, 15, and 16 as strength additive in paper

For application testing of the products synthesized as described in the preceding examples, 0.3% w/v solutions of these products were prepared in the following way: 600 mg of the oxidized product was dispersed in 200 ml tap water. The pH was then adjusted to a value of 5.4 by addition of a drop of concentrated hydrochloric acid. The solution was then poured into a Warring blender equipped with a thermostateable sample container, which was kept on a temperature of 90° C. The solution was mixed at 19500 rpm for ten minutes and was then allowed to cool back to room temperature. The solutions prepared in this way were clear, highly viscous solutions.

Paper making procedure

Pulp was made from a 80/20 Thermomechanical pulp/Softwood mixture (Rygene-Smith & Thommesen TMP225, ex M&M Board Mill, Eerbeek, Netherlands; OULU-pine ECF softwood pulp, Berghuizer Mill, Netherlands). The process water used had 100 ppm CaCO₃ hardness, 50 ppm CaCO₃ alkalinity, and a pH of 7.0-7.5. Water temperature was ambient temperature. The two pulps were refined before mixing on a Hollander beater. TMN was refined at 2.2% consistency for 10 min with 12 kg of weight to a freeness of 47°SR. The softwood pulp was refined at 2.16% consistency for 29 min with 12 kg weight to a freeness of 26°SR. Handsheets were made on a Noble&Wood Handsheet Paper Machine to a grammage of 50 gram per square meter. The pH of the white water was 7-7.5. Dry content of the sheets after the wet press was 32.1%, contact time on the drying cylinder was 41 sec at 105° C., and the final moisture content of the paper was 3.8%. The guar solutions were added to the proportioner of the handsheet machine.

Paper testing

Calliper was measured with the Messmer Büchel Micrometer (model M372200). Tensile strength was measured with a Zwick tensile tester, crosshead speed of 20 mm/min, paper was used in single ply and 15 mm wide. For wet tensile testing, the paper was soaked in demineralized water for 1 min prior to testing. All tests were carried out at 23° C. and 50% relative humidity. The paper was aged for one week under these conditions before testing. Results of the strength test are summarized in the Table 14 below.

TABLE 14

| | | | DRY | WET |
|---|---|---|---|---|
| | ADDITION | GRAMMAGE | TENSILE | TENSILE |
| ADDITIVE | % db | g/m² | kN/m | kN/m |
| blank | — | 50 | 1.39 | 0.05 |
| Example 14 | 0.2 | 54 | 1.58 | 0.05 |
| Example 14 | 0.4 | 52 | 1.83 | 0.24 |
| Example 14 | 0.8 | 52 | 2.01 | 0.32 |
| Example 15 | 0.2 | 52 | 1.62 | 0.14 |
| Example 15 | 0.4 | 52 | 1.59 | 0.19 |
| Example 15 | 0.8 | 50 | 1.77 | 0.22 |
| Example 16 | 0.2 | 51 | 1.54 | 0.15 |
| Example 16 | 0.4 | 51 | 1.61 | 0.18 |
| Example 16 | 0.8 | 50 | 1.74 | 0.24 |

### Example 18

### Dissolution of Oxidized Guar with Varied Temperature and Mixing Time

The experiments described in this example were performed to determine the preferred mixing and temperature conditions for dissolving oxidized cationic guar. The testing is performed with two oxidized cationic guar samples, one having 50% aldehyde groups (Sample A), and one having 35% aldehyde groups (Sample B). Both dried oxidized cationic guar samples were prepared in an 1% cationic guar (N-Hance 3198; Hercules Incorporated, Wilmington Del.) and 1% PEG 6000 (BASF) solution essentially as described in Example 14.

Dried oxidized cationic guar samples were added to tap water to a final concentration of 0.1% (w/v) and mixed in a Warring Blender at mixing position 6 (of 7), at different temperatures (50, 70 and 90 degC). A concentration of 0.1% (w/v) was chosen as this concentration proved to be best suited for SEC analysis as described in Example 20. The percentage of aldehyde groups in these samples was determined using the procedure as described in Example 1.

Subsequent to mixing in the blender samples were filtered through a 0.45 um filter (Schleicher & Schuell, Spartan 13/20) to obtain the dissolved fraction that was analysed with size exclusion chromatography (SEC) to measure the amount of dissolved oxidized cationic guar. Two detectors are connected to the SEC, a refractive index (RI) detector and a viscosity detector. The area of the detected RI peak was chosen as a measure for the amount of dissolved oxidized cationic guar. Mannose concentration as determined by HPAEC-PAD was used to determine the amount of cationic oxidized guar in solution by an independent alternative method (see Example 21).

Table 15 shows how the pH of the sample changes with variations in blending time and temperature.

TABLE 15
pH of Samples A and B After Mixing
with Various Times and Temperatures

| Temperature | Blender Time | Sample A | Sample B |
|---|---|---|---|
| (° C.) | (minutes) | pH | pH |
| 50 | 5 | 8.32 | 7.81 |
| 50 | 10 | 8.5 | 8.26 |
| 50 | 30 | 8.4 | 8.28 |
| 70 | 5 | 8.64 | 8.58 |
| 70 | 10 | 8.63 | 8.62 |
| 70 | 30 | 8.58 | 8.55 |
| 90 | 5 | 9.07 | 9.02 |
| 90 | 10 | 9.05 | 9.09 |
| 90 | 30 | 8.95 | 9.01 |

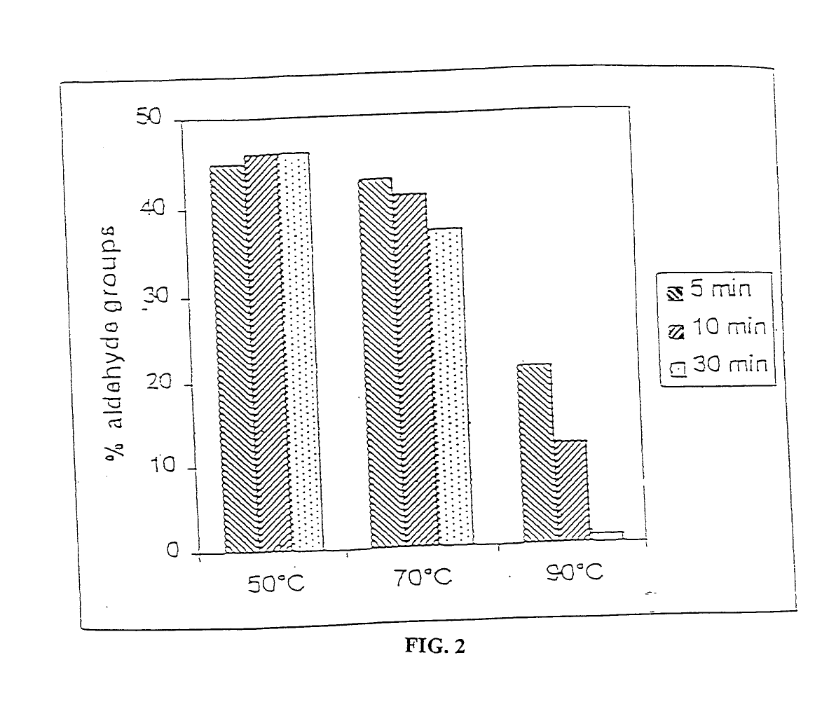

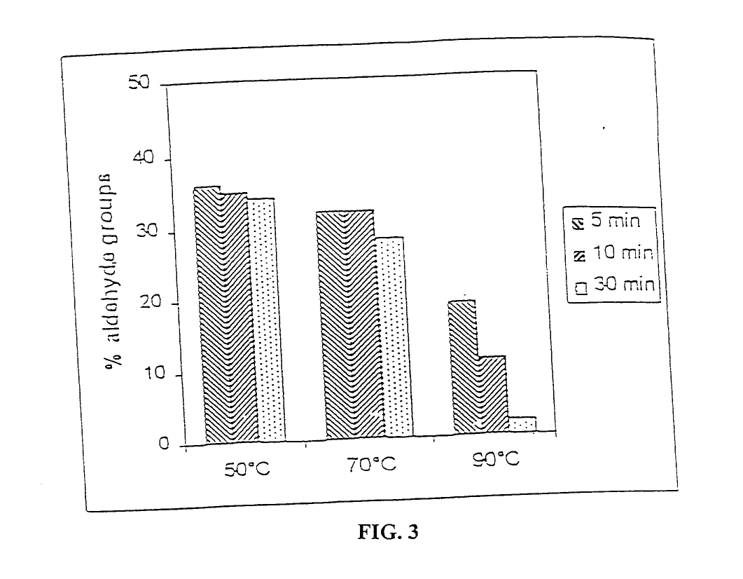

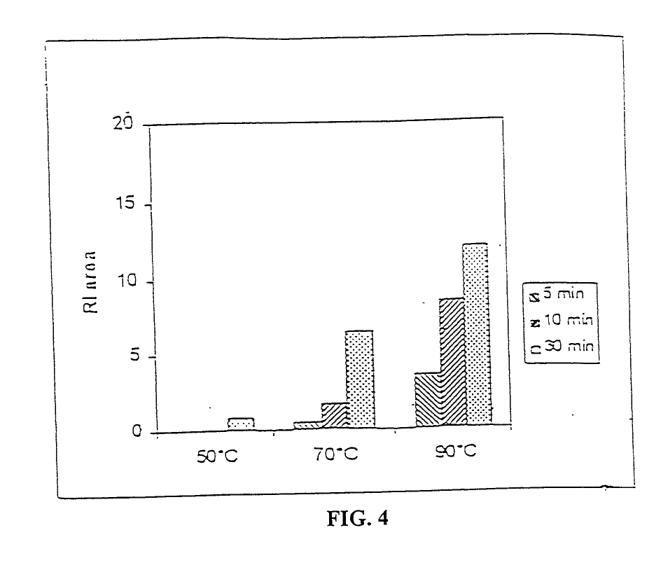

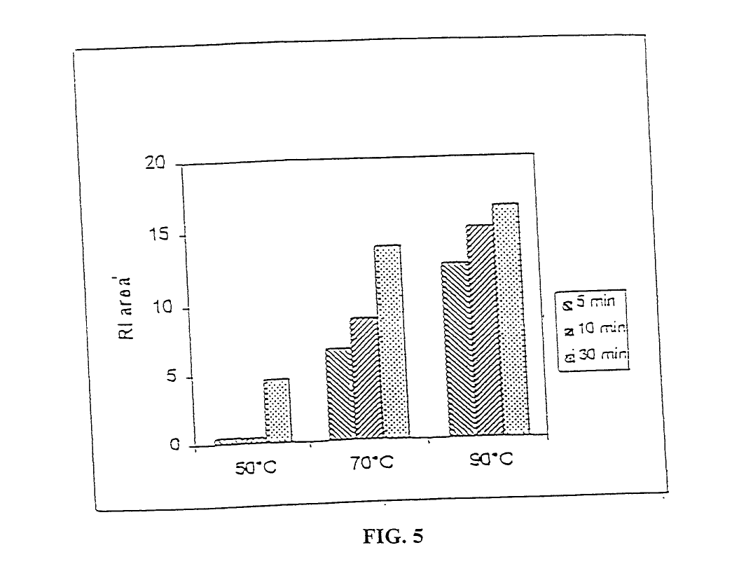

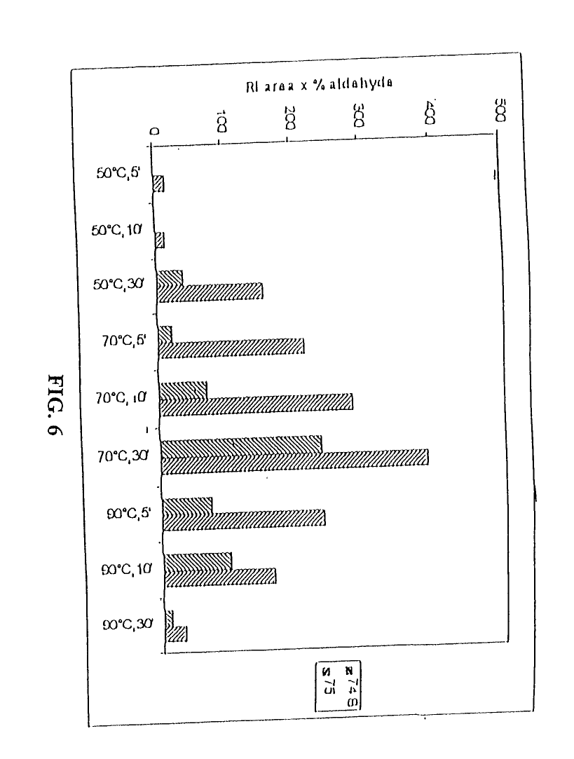

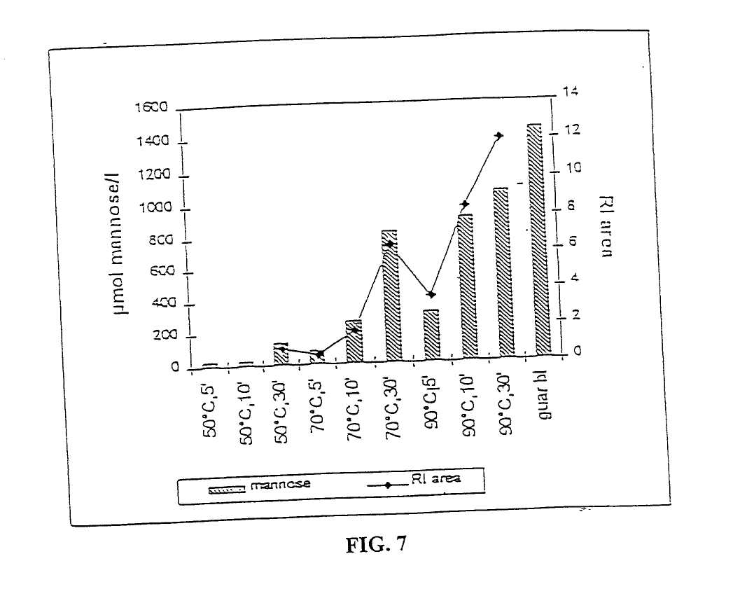

The results (% aldehyde) as determined by the reduction method described in Example 1 for Sample A and B are shown in FIG. 2 and FIG. 3, respectively. The SEC data for Sample A and B are shown in FIG. 4 and FIG. 5, respectively. FIG. 6 shows the product from the RI area with the % aldehyde groups in solution as a function of the blender time and temperature. FIG. 7 shows a comparison of the SEC analysis (as RI area) with the HPAEC analysis (Fmol mannose/L of Sample A). (This comparison was also made for Sample B, but due to the fact that more oxidized guar was dissolved in the sample, the sugar concentration was too high, and the mannose concentration fell out of the standard curve, resulting in an improper measurement.)

The results in FIGS. 2 and 3 show that the higher the temperature, the more guar is dissolved. However, from FIGS. 4 and 5, it is seen that almost no aldehyde groups are left at the higher temperature. Note that the pH of these samples is about 9.0. From the data in FIGS. 2 through 6, it is concluded that 30 minutes in a Warring Blender, at mixing position 6 (of 7), at 70° C., is most favorable. (It should be noted, however, that pH was not controlled in these experiments. The following example (Example 14) shows that controlling the pH results in a change in optimum operating conditions.)

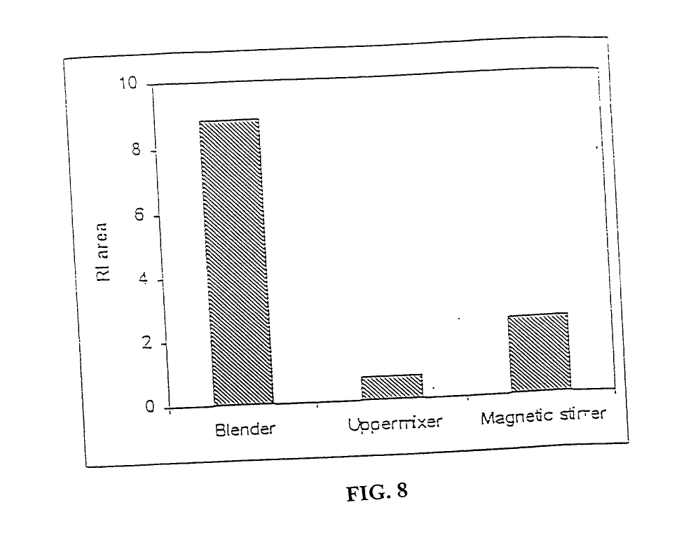

FIG. 7 shows that there is a good comparison between the time consuming HPAEC analysis and the SEC analysis when a concentration of 0.1% oxidized guar is used. However, SEC analysis on a 0.5% oxidized guar solution (dissolved at 70° C. in a blender for 30 minutes) showed a very low RI area. Thus, a sugar analysis is preferred at such high oxidized guar concentrations. To re-confirm the need for a relatively high shear mixer, a simple test was performed. In the simple test, an oxidized guar sample is dissolved using a Warring Blender, a mechanical stirrer, and a magnetic stirrer. Testing conditions are 30 minutes and 70° C. FIG. 8 shows the results of the test, which indicate that the Warring Blender dissolved the oxidized guar to a greater extent than either the mechanical stirrer or the magnetic stirrer.

Thus, from this example, it can be concluded that solubility of cationic oxidized guar is dependent on aldehyde content, temperature, pH, shear, and mixing time of the blender. It appears that, assuming that pH is allowed to vary, the optimal conditions for dissolving a 0.1% cationic oxidized guar sample having 30-35% aldehyde groups in tap water, prepared as 1% guar and 1% PEG is: 70° C., and using a blender for 30 minutes.

### Example 19

### Dissolution of Oxidized Guar with Variations in pH

This example is performed to determine the optimum conditions for dissolving cationic oxidized guar when pH is varied.

In this example, the proper amount of cationic oxidized guar is added to tap water to obtain a 0.1% solution. The pH of the solution is then adjusted with a few drops of M. H.L., while stirring on a magnetic stirrer. The pH-adjusted solution is poured into a Warring Blender which is kept at a temperature of 90° C. The mixing time is varied between 5 and 10 minutes. The sample used is prepared with 1% guar (N-Hance 3198) and 1% PEG 6000.

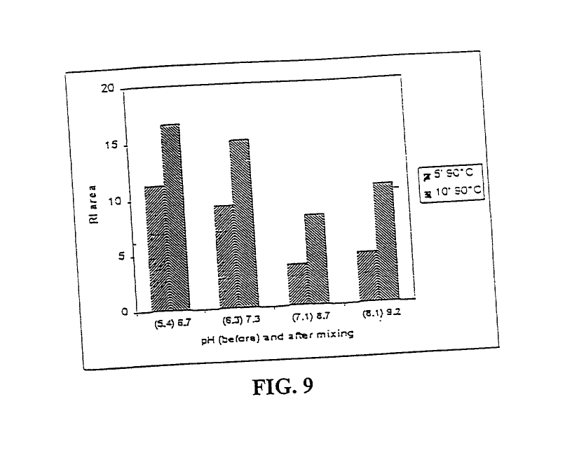

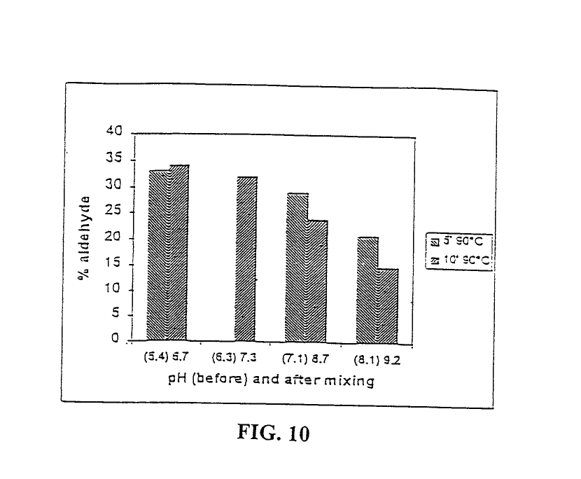

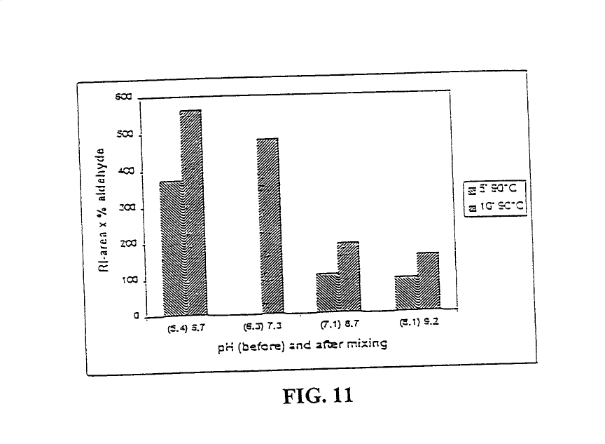

The percent aldehyde groups in the dry product are measured with the reduction method as described in Example 1. After mixing, the samples are analyzed with SEC and the reduction method as described in Example 1. The RI area data that are generated with SEC are used as a measure for the dissolved cationic oxidized guar. The percent aldehyde groups after dissolution is measured with the reduction method as described in Example 1. FIG. 9 shows the RI areas for a 0.1% cationic oxidized guar sample having 35% aldehyde groups, dissolved in tap water, with various pH and mixing times, with a mixing temperature of 90° C. FIG. 10 shows the percent aldehyde groups of a 0.1% sample (with 35% aldehyde groups), dissolved in tap water, with various pH and mixing times, with a mixing temperature of 90° C. (The analysis of this sample dissolved at a pH of 6.3 and mixed for 10 minutes failed, so this data is not presented.) FIG. 11 shows the product of the RI area and the percent aldehyde groups, given at various pH and mixing times, -with a mixing temperature of 90° C.

From this example, it can be concluded that acidifying the sample in tap water with a drop of acid seems to protect the aldehyde groups of the dissolved cationic oxidized guar during the mixing at high shear and temperature of 90° C. There is a dramatic decrease in the percent aldehyde groups on the dissolved cationic oxidized guar when the pH is greater than 7. There is also a large difference in the dissolution of the cationic oxidized guar between 5 minutes and 10 minutes mixing. Longer mixing time appears to dissolve more of the cationic oxidized guar without affecting the percent of aldehyde groups.

Thus, it appears that, when pH, temperature, and mixing time, are considered, the optimum conditions for dissolving cationic oxidized guar are: 1) dissolve the oxidized guar in tap water acidified to a pH of 5.4, 2) using a high shear and intensive turbulence blender (Warring Blender) at a temperature of 90° C., mix for 10 minutes.

### Example 20

### Measurement of dissolved guar by size exclusion chromatography (SEC)

The SEC analyses were performed on a Hewlett Packard 1050 system with vacuum degasser. the system was equipped with a TSK-gel column set: PWXL guard, G2500PWXL and G300OPWXL (TOSOHAAS). The temperature of the column oven was 40 C. The eluent was a 0.1 M acetic acid (Merck) solution with the pH adjusted to 4.4 with sodium hydroxide (Baker, 7067). 100 μL sample was injected. Separation was performed at a flow rate of 0.8 mL/min. The compounds were detected by a 90 degrees laser light scattering detector (Viscotek model T60A), a viscosity detector (Viscotek model T60A) and a Refractive Index detector (Hewlett Packard 1047A). The refractive index area of the oxidized guar peak was calculated by the Viscotek software and used as a relative number for the determination of the amount of polymer in solution. The areas were compared with the amount of mannose present in the sample.

### Example 21

### Measurement of dissolved guar by HPAEC-PAD

Mannose content in the filtrates was determined by using HPAEC-PAD in combination with methanolysis and TFA hydrolysis. 250 μL sample (filtrate) was pipetted into a screw-cap test tube and the sample was dried by N2 gas evaporation. The dried sample was first hydrolyzed by adding 0.5 ml of a 2 M methanolic H.L. solution (Supelco, 3-3050) under nitrogen The tubes were closed and incubated at 80° C. for 16 hours using an oil bath. After cooling, the samples were dried under a nitrogen gas flow. A second hydrolysis step was performed by adding 0.5 ml of a 2 M trifluor acetic acid solution (Acros, 13972-1000). The samples were heated to 121 C and incubated for 1 hour. After cooling, the samples were evaporated to dryness using a nitrogen gas flow. The samples were dissolved in 200 AL acetate buffer (0.05 M sodium acetate, pH=5), put into a vial and subjected to HPAEC analysis. A calibration line of mannose (Acros, 15.060.0250) was made for quantification. Five different aliquots of a stock solution of 14.9 mg mannose (99%) in 200 ml water were subjected to the same hydrolysis steps as the samples. The volumes of the standard mannose solution were: 200, 100, 70, 40 and 10,μL corresponding to a final concentrations of 409.3, 204.7, 143.3, 81.9 and 20.5 3 mol/L of mannose, respectively.

The HPAEC equipment consists of a GP40 gradient pump, an AS3500 autosampler and an ED40 electrochemical detector (PAD) with a gold electrode (Dionex, Breda, Netherlands). 20 μL of sample was injected at room temperature on a CarboPac PA1 column (Dionex). Separation was performed with a flow rate of 1 mL/min using a combined gradient of three eluents prepared from milli Q water (Millipore). Eluent A: 0.1 M NaOH prepared from a 50% solution of NaOH (Baker, 7067). Eluent B: 0.1 M NaOH and m. sodium acetate (Merck, 1.06268.1000). Eluent C: milli Q water. The eluents were degassed by helium. The following gradient was applied for NaOH: 0-20 min, 20 mM NaOH; 20-35 min, 100 mM NaOH; 35-50 20 mM NaOH. The simultaneous gradient of NaAc was: 0-21 min, 0 M; 21-30 min, 0-300 mM; 30.01-35 min, 1000 mM NaAc; 35.01-50 min, 0 M.

The effluent was monitored using a pulsed-electrochemical detector in the pulsed amperometric mode (PAD) with a gold working electrode and an Ag/AgCl reference electrode (Dionex) to which potentials of E1 0.1 V, E2 0.65 V and E3 BO.1 V were applied for duration times of T1 0.4 s, T2 0.2 s, T3 0.4 s. Data collection was done with Peaknet software release 4.2 (Dionex).

From the amount of mannose, present in the sample, the amount of oxidized guar can be calculated if the ratio of galactose and mannose is known. Analysis of guar-derivatives by the reduction method described in Example 1 show that the ratio is close to 1:2.

### Example 22.

This example is directed to the illustration of having both catalase and peroxidase present in the oxidation of guar gum by galactose oxidase. The guar gum was enzymatically degraded to low-molecular weight prior to the oxidation. The low-molecular weight of the guar allowed for the oxidation reaction to take place at signifiantly higher solids concentration.

Three samples were compared side by side:

Sample 1: With galactose oxidase, catalase, and peroxidase.

Sample 2: With galactose oxidase and catalase.

Sample 3: With galactose oxidase only.

Neutral guar gum SUPERCOL G2S (Hercules, Inc. Delaware, USA) was used.

The guar was hydrolyzed under the following conditions:

0.0075 part of mannanase (from ChemGen, Corp. Maryland, USA) was added to 95 parts of water at 60° C. Without delay and while stirring with an overhead mixer, 5 parts of the guar gum was sprinkled into the water within 10 minutes. The reaction was allowed to proceed for about 60 minutes to about 55 cps of Brookfield viscosity (at 25° C., 30 rpm with spindle #31, and a small sample adapter #13 R vessel). The mannanase was deactivated by rapidly heating to 90° C. within 10 minutes using live steam through the jacket of the reactor, and then held at 90° C. for 30 minutes. The reaction mixture was then cooled to 25° C.

The low molecular weight guar was then oxidized under the following conditions:

500 g of the low-molecular-weight guar gum hydrolyzates solution at 5% solids was held at 25° C. in a glass reactor with an overhead stirrer. The solution was sparged with air at 0.1 volume of air per volume of the guar solution per minutes (vvm), while continually stirring at 200 rpm. 160 units of galactose oxidase (BioTechnical Resources, Wisconsin, USA), 600 units of catalase Terninox Ultra 50 L (Novo.Nordisk, Denmark), and 15 units of peroxidase NS5 1004 (Novo Nordisk, Denmark) per gram of guar hydrolyzates was added for Sample 1. The proxidase was omitted for Sample 2. Both peroxidase and catalase were omitted for Sample 3. The reaction was allowed to proceed for about 4 hours. The enzymes were deactivated by lowering the pH to 4.0 using 0.5N H₂SO₄.

The final average molecular weight range of the oxidized guar product was approximately 56,000 Daltons, and the extent of oxidation was determined using a HPLC method.

The following Table 16 shows the degree of the aldehyde conversion among the three samples.

TABLE 16

| Sample | Remark | Aldehyde % |
|---|---|---|
| Sample 1 | With all three enzymes | 36.9 ± 0.6 |
| Sample 2 | Without peroxidase. | 4.9 ± 0.8 |
| Sample 3 | With galactose oxidase only | 2.8 ± 0.2 |

It is seen that significant aldehyde conversion can be achieved when all three enzymes are present in the oxidation reaction.

From the foregoing descriptions, one skilled in the art can easily ascertain the essential characteristics of this invention, and without departing from the spirit and scope thereof, can make various changes and modifications of the invention to adapt it to various usages and conditions.

What is claimed is:

1. A composition comprising (a) galactose oxidase; (b) galactose containing polysaccharide (c) one electron oxidant; (d) hydrogen peroxide remover; and (e) aqueous solvent.

2. The composition according to claim 1, wherein the one electron oxidant comprises an enzyme.

3. The composition according to claim 1, wherein the hydrogen peroxide remover comprises catalase.

4. The composition according to claim 2, wherein the enzyme comprises at least one of peroxidase and laccase.

5. The composition according to claim 4, wherein the enzyme comprises peroxidase and the peroxidase comprises soybean peroxidase.

6. The composition of claim 4, wherien the enzymne comprises peroxidase and the perosidase comprises horseradish peroxidase.

7. The composition of claim 6, wherein the hydrogen peroxide remover comprises catalase.

8. The composition according to claim 7, wherein the galactose containing polysaccharide comprises at least one of carbohydrate gums, pectins and cellulosics.

9. A composition according to claim 8, wherein the galactose containing polysaccharides comprises carbohydrate gums and wherein the carbohydrate gum comprises at least one of polygalactomannan gums or their ether derivatives, arabinogalactan gums or their ether derivatives, galactoglucomannan hemicelluloses or their ether derivatives, carubin, lichenan, tamarind and potato galactan, polygalactoglucans, polygalactoglucomannans and polygalactan gums.

10. A composition according to claim 9, wherein the carbohydrate gum comprises polygalactomannan gum and wherein the polygalactomannan gum comprises at least one of locust bean gum, guar gum, tamarind gum, gum arabic, tara and fenugreek.

11. A composition according to claim 10, wherein the polygalactomannan gum comprises guar gum.

12. A composition according to claim 9, wherein the carbohydrate gum comprises polygalactan gum and wherein the polygalactan gum comprises at least one of carrageenans and alginates.

13. The composition of claim 1, wherein the one electron oxidant comprises a chemical oxidant.

14. The composition of claim 1, wherein the chemical oxidant comprise at least one of ferricyanide, H₂IrCl₆, [Co(phen)₃]³⁻, [Co(dipic)₂]⁻.

15. The composition of claim 14, wherein the one electron oxidant comprises ferricyanide and the hydrogen peroxide remover comprises catalase.

16. The composition of claim 1, wherein the polysaccharide comprises guar; the one electron oxidant comprises horseradish peroxidase and the hydrogen peroxide remover comprises catalase.

17. The composition of claim 16, wherein the composition solid.

18. The composition of claim 17, wherein the composition is re-solubilized.

19. The composition of claim 1, wherein the composition further comprises paper fiber.

20. The composition of claim 1, wherein the composition further comprises natural or synthetic polymers.

21. The composition of claim 1, wherein the composition further comprises plasma.

22. A process for oxidizing a galactose oxidase substrate containing at least one alcohol group convertible to an aldehyde in an industrial application comprising reacting, in an aqueous solvent, the substrate, galactose oxidase, one electron oxidant capable of activating the galactose oxidase and hydrogen peroxide remover, under conditions to oxidize the galactose oxidase substrate.

23. The process of claim 22, wherein the hydrogen peroxide remover comprises catalase.

24. The process of claim 23, wherein the galactose oxidase substrate comprises a polysaccharide.

25. The process of claim 24, wherein the polysaccharide comprises at least one of carbohydrate gums, pectins and cellulosics.

26. The process of claim 25, wherein the polysaccahride comprises carbohydrate gum and the carbohydrate gum comprises at least one of polygalactomannan gums or their ether derivatives, arabinogalactan gums or their ether derivatives, galactoglucomannan hemicelluloses or their ether derivatives, carubin, lichenan, tamarind and potato galactan, polygalactoglucans, polygalactoglucomannans and polygalactan gums.

27. A composition according to claim 26, wherein the carbohydrate gum comprises polygalactomannan gum and wherein the polygalactomannan gum comprises at least one of locust bean gum, guar gum, tamarind gum, gum arabic, tara and fenugreek.

28. A composition according to claim 27, wherein the polygalactomannan gum comprises guar gum.

29. A composition according to claim 26, wherein the carbohydrate gum comprises polygalactan gum and wherein the polygalactan gum comprises at least one of carrageenans and alginates.

30. The process of claim 22, wherein the one electron oxidant comprises an enzyme.

31. The process of claim 30, wherein the enzyme comprises at least one of peroxidase and laccase.

32. The process of claim 22, wherein the carbohydrate gum comprises guar, the one electron oxidant comprises soybean peroxidase and the hydrogen peroxide remover comprises catalase.

33. The process of claim 30, wherein the galactose oxidase substrate comprises a paper strength additive.

34. The process of claim 30, wherein the substrate comprises a binding agent for use in the paper industry.

35. The process of claim 22, wherein the carbohydrate gum comprises guar, the one electron oxidant comprises horseradish peroxidase and the hydrogen peroxide remover comprises catalase.

36. The process of claim 22, wherein the one electron oxidant comprises a chemical oxidant.

37. The process of claim 36, wherein the chemical oxidant comprises at least one of ferricyanide, H₂IrCl₆, [Co(phen)₃]³⁻, [Co(dipic)₂]⁻.

38. The process of claim 37, wherein the one electron oxidant comprises ferricyanide and the hydrogen peroxide remover comprises catalase.

39. The process of claim 22, further comprising drying the oxidized composition.

40. The processes of claim 39, further comprising re-solubilizing the oxidized composition.
